SEQUENCE LISTING (6 sequences)

SEQ ID NO: 1 - DNA, 66 bp, Homo sapiens
ggcggcgcggagttctcggctcgctccaggaagaggaaggcaaacgtgaccgtttttttgcaggat

SEQ ID NO: 2 - protein, 22 aa, Homo sapiens
GGAEFSARSRKRKANVTVFLQD

SEQ ID NO: 3 - protein, 22 aa, Mouse
GGSDLSVRSRKRKANVAVFLQD

SEQ ID NO: 4 - protein, 22 aa, Rat
GGSDLSVRSRKRKPNVPVFLQD

SEQ ID NO: 5 - protein, 22 aa, chicken
GGAEGTVRARKRKADVATFLQD

SEQ ID NO: 6 - protein, 22 aa, Artificial Sequence
  Synthetic
FAFGRQVNKARSEKALGVSDRT

(19) United States
(12) Patent Application Publication
(10) Pub. No.: US 2011/0105382 A1
(43) Pub. Date: May 5, 2011

(54) CALMODULIN-BINDING PEPTIDES THAT REDUCE CELL PROLIFERATION IN CANCER AND SMOOTH MUSCLE PROLIFERATION DISEASES

(76) Inventors: Mansoor Husain, Toronto (CA); Jaehyun Choi, Toronto (CA)

(21) Appl. No.: 12/674,368

(22) PCT Filed: Aug. 19, 2008
(86) PCT No.: PCT/CA2008/001474

§ 371 Date: Jun. 2, 2010

Related U.S. Application Data
(60) Provisional application No. 60/957,361, filed on Aug. 22, 2007.

(51) Int. Cl. A61K 38/17; C07K 14/47; C07K 7/08; C07K 7/06; C07K 19/00; C12N 5/09; C12N 5/071; A61P 35/00; A61P 9/10; A61P 9/12; C07H 21/00; C07K 16/18; A61P 21/00; A61P 17/00; A61P 1/00; A61P 13/10; A61P 13/00; A61P 15/00

(52) U.S. Cl. 514/1.9; 530/326; 530/327; 530/328; 530/329; 530/330; 530/324; 514/21.4; 435/375; 514/19.3; 514/15.7; 536/23.5; 530/387.9; 514/18.6

(57) ABSTRACT

The invention relates to an isolated peptide comprising all or part of the amino acid sequence: GGAEFSARSR KRKANVTVFL QD (SEQ ID NO: 2), wherein the peptide reduces cell proliferation. The invention also includes variants, such as SEQ ID NO:3-5 and fragments of at least 5 amino acids of SEQ ID NO:2-5 that reduce cell proliferation. The peptides are useful for treating cancer and diseases characterized by smooth muscle proliferation, such as restenosis.

### FIELD OF THE INVENTION

The invention relates to peptides for use in treatment of cancer and vascular disease.

### BACKGROUND OF THE INVENTION

There are a number of diseases caused by cell proliferation which are not currently treated properly and safely with conventional medicine. For example, cancer is caused by cells that proliferate uncontrollably. Radiation treatment and surgery are often used for cancer treatment even though these treatments produce severe side effects. Despite recent advances in cancer chemotherapy, radiation treatment and surgery remain the mainstays of cancer treatment. Cell cycle inhibitors are an example of a type of chemotherapy drug that have been used to treat cancer, but these compounds have been shown to cause severe toxicity with poor selectivity.

Vascular smooth muscle cell proliferation is responsible for a number of diseases, such as restenosis following balloon angioplasty. Restenosis, is a relatively frequent (−10%) consequence of balloon angioplasty procedures performed on occluded or narrowed coronary arteries and/or coronary artery bypass grafts (“CABG”). Angioplasty has over-taken CABG surgery as the most common heart procedure performed in the world today but current therapies have had limited success in preventing restenosis. As well, concerns surrounding the safety of radioactive stents, and stents that elute chemotoxic agents, highlight the need for alternative strategies aimed at treating this disease.

Advances have been made in understanding the biochemical interactions that regulate cell division. Calmodulin (“CaM”), a small acidic protein of 16.7 kDa (Genbank accession no. for human CaM is CAA36839), is a principal Ca2+ sensor in eukaryotic cells that contains four EF-hand Ca2+ binding motifs. Upon binding with Ca2+, Ca2+/CaM performs a large conformational change of its highly flexible α-helical linker resulting in its binding to a variety of target proteins. These are collectively termed CaM-binding proteins, through which Ca2+-sensitivity is expressed in a variety of cell biological functions including ion channel function, gene regulation, smooth muscle contraction, and cell motility. A role of CaM as a regulator of cell cycle progression has also been recognized. A direct interaction between the major calcium {Ca2+} signal-transducing protein calmodulin (CaM), and the critical regulator of cell cycle progression cyclin E, is necessary for Ca2+-sensitive cyclin E/CDK2 activity during G1 to S phase cell cycle progression in vascular smooth muscle cells (“VSMC”). The human cyclin E gene was cloned, sequenced and functionally characterized as a cell cycle regulatory protein between 1991 and 1992 (Lew Cell 91; Koff Cell 91; Koff Science 92; Dulic Science 92; Matsushime Cell 1991); Genbank accession no. for human cyclin E is NP 476530). This metabolic interaction is related to proliferation of vascular smooth muscle cells and cancer cells, however, there are no medicines which target this interaction selectively in order to treat proliferative disorders.

### SUMMARY OF THE INVENTION

The invention relates to isolated peptides for use in treatment of cancer and vascular disease by disrupting calmodulin binding to cyclin E protein. The inventors have synthesized useful isolated peptides with the sequence shown in FIG. 9 (SEQ ID NOS:2-5) as well as shorter peptides based on this sequence. The inventors provide isolated peptides as small as 5 amino acids that provide cell proliferation inhibition activity (“CBS activity”).

The peptides of the invention reduce proliferation of primary vascular smooth muscle cells and cancer cells by causing cell cycle arrest. They do not cause cytotoxicity (i.e. cell death) and do not interfere with other CaM-signaling pathways—they act selectively through the CaM-cyclin E interaction. These characteristics set the peptides apart from other existing cell cycle inhibitors and make them useful for therapeutic treatments that inhibit cell proliferation. CBS peptides have several specific advantages, including (a) high selectivity; working in a cyclin E-specific/dependent manner, and (b) low toxicity; not causing cell death and not inhibiting basal CDK2 activity.

The invention relates to isolated peptides capable of reducing cell proliferation and comprising all or part of (SEQ ID NOS:2-5). In one embodiment, the peptide reduces smooth muscle cell or cancer cell proliferation. The isolated peptides are optionally synthetic peptides or recombinant peptides.

In another embodiment, the peptide has three consecutive hydrophobic residues located within five amino acids of the C-terminus. Optionally, at least one of the hydrophobic residues comprises a leucine residue. Optionally, the leucine residue is located within 5 amino acids or within 3 amino acids of the C-terminus.

In a variation of the invention, the peptide comprises a truncated amino acid sequence of the peptide of any one of (SEQ ID NOS:2-5) or a fragment of the amino acids of the peptide of (SEQ ID NO:2). Optionally, the truncated peptide contains amino acids 2-21, 3-20, 4-19, 5-18, 6-17, 7-16, 8-15, 9-14, 12-20, 14-20 or 16-20 of the peptide of (SEQ ID NOS:2-5). The fragment optionally contains 5-10, 10-15, 15-20 or 20-22 amino acids of the peptide of (SEQ ID NOS:2-5). In another variation, the peptide comprises a sequence falling within the formula V-X1-X2-F-L, wherein X1 is optionally a hydrophobic amino acid or a neutral amino acid (eg. selected from the group of V, I, L, F, W, C, A, Y, H, T, S, P, G, R or K), typically T, A, P and wherein X2 is optionally a hydrophobic amino acid or a neutral amino acid (eg. selected from the group of V, I, L, F, W, C, A, Y, H, T, S, P, G, R or K), typically V or T. The peptide optionally contains 5-10, 10-15, 15-20 or 20-22 or 23-50 amino acids.

In another variation of the invention, the peptide comprises at least 5, 6, 7, 8, 9 or 10 amino acids of the peptide of any one of SEQ ID NOS:2-5.

In another variation of the invention, the peptide contains at least 80% sequence identity with the amino acid sequence of SEQ ID NOS:2-5 and reduces cell proliferation. For example, the peptides having identity optionally contain all or part of the amino acid sequences of any of SEQ ID NOS:2-5.

In another embodiment of the invention, the peptide of the invention further comprises a TAT linker amino acid sequence, comprising all or part of RRRQRRKKRG, to increase the uptake of the peptide into a cell.

In another embodiment of the invention a pharmaceutical composition comprises any of the peptides of the invention and a carrier.

The peptides may be modified as described below to produce variants of the peptide that have desired activities. For example, the invention includes peptide sequences plus or minus amino acids at the amino and/or carboxy terminus of the peptide sequences. In another embodiment, the invention includes fusion proteins, comprising the CBS peptide or labeled CBS peptide. A peptide of the invention may include various structural forms of the primary CBS peptide which retain biological activity. For example, a peptide of the invention may be in the form of acidic or basic salts or in neutral form. In addition, individual amino acid residues may be modified by oxidation or reduction.

The invention further relates to a method of reducing cell proliferation caused by cyclin E specific calcium/calmodulin dependent CDK2 activity in the cell, comprising administering to the cell a peptide or pharmaceutical composition of the invention. Optionally, the cell is a cancer cell or a smooth muscle cell. The cancer cell is optionally a cervical cancer cell, an osteosarcoma cancer cell or a lung cancer cell.

The invention further relates to the use of the peptide or the pharmaceutical composition of the invention for treatment of cancer and to the method of treatment of cancer in a mammal wherein the peptide or pharmaceutical composition is administered to the mammal. Optionally, the cancer comprises cancer cells undergoing calcium sensitive cyclin E protein mediated cell proliferation. The cancer is optionally cervical cancer, osteosarcoma or lung cancer.

The invention further relates to the use of the peptides or the pharmaceutical composition of the invention for reducing proliferation of vascular smooth muscle cells and the method of reducing proliferation of vascular smooth muscle cells in a mammal in need thereof wherein the peptide or pharmaceutical composition is administered to the mammal. Optionally, the disorder comprises vascular smooth muscle cells undergoing calcium sensitive cyclin E protein mediated cell proliferation. Optionally, the peptide or pharmaceutical composition inhibits CDK2 activity by inhibiting the binding of calmodulin to cyclin E protein.

The invention further relates to the use of the peptides or the pharmaceutical composition of the invention for the treatment of a vaso-occlusive disorder and to a method of treatment of a vaso-occlusive disorder in a mammal in need thereof wherein the peptide or pharmaceutical composition is administered to the mammal. Optionally, the vaso-occlusive disorder comprises restenosis, Burger syndrome, atherosclerosis, scleroderma, Raynauds disease, hypertension pulmonary hypertension or post-vascular surgery smooth muscle cell proliferation. The atherosclerosis optionally comprises coronary artery disease, peripheral artery disease or cerebrovascular disease. The hypertension optionally is caused by smooth muscle cell proliferation after vascular surgery. The vascular surgery optionally consists of coronary angioplasty, coronary stent placement, coronary by-pass surgery, peripheral stent placement, vascular grafting, thrombectomy, vascular angioplasty, and vascular stenting. Optionally, the disorder comprises vascular smooth muscle cells undergoing calcium sensitive cyclin E protein mediated cell proliferation. Optionally, the pharmaceutical composition is a stent.

The invention further relates to the use of the peptides or the pharmaceutical composition for the treatment of a visceral smooth muscle cell disorder and a method of treatment of a visceral smooth muscle cell disorder in a mammal in need thereof wherein the peptide or pharmaceutical composition is administered to the mammal. Optionally, the visceral smooth muscle cell disorder comprises inflammatory bowel disease, bowel strictures, spastic bladder, urinary retention and uterine cramps. The smooth muscle cell proliferation is optionally caused by vascular injury. Optionally, the disorder comprises vascular smooth muscle cells undergoing calcium sensitive cyclin E protein mediated cell proliferation.

The invention further relates to an isolated nucleic acid comprising all or part of (SEQ ID NO:1) that encodes a peptide of the invention. The invention further relates to an isolated nucleic acid encoding all or part of any of SEQ ID NOS:2-5. The encoded peptide reduces smooth muscle cell or cancer cell proliferation.

The invention further relates to an isolated antibody that selectively binds any one or more of the peptides of the invention.

The invention further relates to a stent comprising any of the peptides or the pharmaceutical composition of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from the detailed description.

### BRIEF DESCRIPTION OF THE DRAWINGS

Embodiments of the invention will be described in relation to the drawings in which:

FIG. 1 shows that the CaM-cyclin E interaction is reduced by the synthetic peptide of CaM-binding motif.

FIG. 1A provides the sequences of the synthesized peptides. Human CBS 22 mer (SEQ ID NO:2): Isolated peptide corresponds to sequence of the CaM binding region in human cyclin E1. 5A: Same as human CBS 22 mer except for 5 alanine mutations at every hydrophobic residue. NC: negative control which has the same length, 22 amino acids, as CBS (SEQ ID NO:2).

FIG. 1B shows a histone H1 in vitro kinase assay from G₁/S-synchronized mouse VSMC in the presence of tested peptides (each 100 μM).

FIG. 1C shows the level of cyclin E/CDK2 complex formation and CaM-cyclin E interaction in the presence of each peptide (100 μM) by co-immunoprecipitation analysis.

FIG. 1D shows a histone H1 in vitro kinase assay from G₁/S-synchronized mouse VSMC with differing Ca²⁺ concentrations in the presence of CBS (SEQ ID NO:2) or NC (both 100 μM).

FIG. 2 shows the effects of the CBS peptide (SEQ ID NO:2) on VSMC proliferation.

FIG. 2A shows confocal microscopy images (60×) of mouse VSMC after nucleofection of FITC tagged-CBS or -NC (both 100 μM). Green: FITC, Blue: Hoechst.

FIG. 2B shows the level of proliferation of primary mouse aortic SMC (MTT assay) in the presence of CBS (SEQ ID NO:2) or NC peptides.

FIG. 3 shows the mechanism of CBS-induced (SEQ ID NO:2) proliferation reduction.

FIG. 3A shows a Lactate dehydrogenase (LDH) assay after CBS (SEQ ID NO:2) and NC peptide delivery (1 mM) into asynchronized primary mouse aortic SMC.

FIG. 3B depicts the cell cycle analysis after delivery of CBS (SEQ ID NO:2) or NC peptides (1 mM) into starved (G₀-phase cell cycle synchronized) and then serum-stimulated primary mouse aortic SMC.

FIG. 3C shows western blotting after delivery of the CBS (SEQ ID NO:2) or NC peptides. Representative blots from three separate experiments are shown with normalized band intensities.

FIG. 3D shows a MTT cell proliferation assay comparing the CBS (SEQ ID NO:2), scramble peptide control (sequence: FAFGRQVNKARSEKALGVSDRT (SEQ ID NO:6)) and NC peptide. All peptides were nucleofected at the same concentration of 1 mM. *P<0.05.

FIG. 4 shows that CBS reduces the proliferation of VSMC in a cyclin E-specific/dependent manner.

FIG. 4A shows western blotting from wild type- (WT) and cyclin E1/E2 double knock out- (KO) mouse embryonic fibroblasts (MEFs).

FIG. 4B is a MTT assay showing the level of proliferation of WT- & KO-MEFs in the presence (1 mM) or absence of peptide delivered into asynchronized cells. *P<0.05.

FIG. 4C reveals the cell cycle analysis of WT- & KO-MEFs after treatment of calmidazolium (CMZ, nonselective CaM inhibitor) or peptides (CBS (SEQ ID NO:2) or NC). Each treatment was performed on starved cells followed by serum stimulation with 10% FBS for 20 hr (for CMZ treatment) or 24 hr (for peptide treatment via nucleofection). *P<0.05, **P<0.01

FIG. 5 shows the results of treatment of TAT-CBS (SEQ ID NO:2)-His peptide to VSMC.

FIG. 5A shows the sequence of TAT-CBS (SEQ ID NO:2)-His peptide. 10 amino acids from the TAT domain of HIV-1 were fused to the N-terminus of CBS (SEQ ID NO:2). 6×-His tag was also fused at the C-terminus.

FIG. 5B shows western blotting after TAT-CBS-His or TAT-NC-His peptide treatment (both 100 μM) to primary mouse aortic SMC. Peptides were added to serum free DMEM cell culture media for 1 hr.

FIG. 5C is a MTT assay showing the level of proliferation of primary mouse aortic SMC in the presence TAT-CBS (SEQ ID NO:2) and TAT-NC (both 100 μM). Peptides were added to serum free DMEM cell culture media for 1 hr followed by fresh media replacement with 10% FBS. MIT assay was performed after 2 days. Relative OD intensity was obtained by normalizing each intensity to control group without peptide. **P<0.01.

FIG. 5D shows the dose-response curve and IC₅₀ measurement of TAT-CBS (SEQ ID NO:2) for the inhibitory effects on primary VSMC proliferation. MTT assay was performed 2 days after peptide treatment. An IC₅₀ value was calculated by nonlinear regression analysis based on a Boltzmann sigmoid curve from 3 separate experiments.

FIG. 5E shows the proliferation of three human cancer cells—HeLa (human cervical cancer cell), Saos-2 (human osteosarcoma), and A549 (human lung cancer cell)—in the presence TAT-CBS-His (100 μM). Relative OD intensity was obtained by normalizing each intensity to control group without peptide. **P<0.01 vs. No pep.

FIG. 6 depicts an in vivo application of TAT-CBS to a mouse carotid artery injury model.

FIG. 6A shows images (10×) of H&E stained injured and uninjured mouse carotid arteries with and without treatment of TAT-CBS (SEQ ID NO:2)-His or TAT-NC-His.

FIG. 6B shows media mass (μm²) and Intima/Media (UM) ratios of mouse common carotid arteries harvested at 14 days following injury. All injuries were performed on the right side, while the left common carotid artery of each mouse was used as an uninjured control. *P<0.05, **P<0.01

FIG. 6C shows representative images of PCNA staining (20×).

FIG. 6D shows the percentage of PCNA-positive nuclei at 14 days following injury. **P<0.01

FIG. 7 shows a small motif in CBS.

FIG. 7A is a MTT assay with TAT-truncated peptides with primary mouse aortic SMC, and human cancer cells HeLa, Saos-2, or A549. All peptides were employed at the concentration of 100 μM, and the MTT assay was performed 2 days later. The sequence of each peptide is shown in Table 1A.

FIG. 7B is a histone H1 kinase assay with 7 mer peptides. Each peptide (200 μM) was added during the immunoprecipitation step with cyclin E antibody. Sequence of each peptide is shown in Table 1B. *P<0.05 vs. No pep.

FIGS. 7C & D shows MTT assays with additional TAT-truncated peptides with primary mouse aortic SMC, and human cancer cells HeLa, Saos-2 or A549. All peptides were employed at the concentration of 100 μM, and the MTT assay was performed 2 days later. The sequence of each peptide is shown in Table 1C, and 1D, respectively.

FIG. 8 shows an in vivo application of truncated peptides to mouse carotid artery injury model.

FIG. 8A shows media mass (μm²) and FIG. 8B shows Intima/Media (I/M) ratios of mouse common carotid arteries harvested at 14 days following injury. All injuries were performed on the right side, while the left common carotid artery of each mouse was used as an uninjured control. *P<0.05, **P<0.01

FIG. 9 shows the isolated nucleic acid sequence of SEQ ID NO: 1 and the amino acid sequence of SEQ ID NO:2, SEQ ID NO: 3, SEQ ID NO: 4, and SEQ ID NO: 5. Optionally, SEQ ID NO:2 is obtained from human, SEQ ID NO: 3 is obtained from mouse, SEQ ID NO: 4 is obtained from rat, and SEQ ID NO: 5 is obtained from chicken.

FIG. 10 shows the anti-proliferative effect of TAT-CBS using tritiated-thymidine assays in primary human aortic VSMC (FIG. 10A), MEF (FIG. 10B) and Cyclin E1/2 KO MEF (FIG. 10C) treated with 10 and 100 μM of TAT-CBS, TAT-NC or TAT-Scramble for 1.0 h at 37° C. prior to the start of the thymidine incorporation assay. Compared to controls, TAT-CBS inhibited 3H-thymidine incorporation in primary human aortic SMC in vitro

FIG. 10A shows a dose-dependent, anti-proliferative effect of TAT-CBS 72 h after treatment in human aortic VSMC compared to control cells treated with TAT-NC (n=3, each condition was done in triplicate and average values are reported). *P<0.05

FIG. 10B shows a dose-dependent, anti-proliferative effect of TAT-CBS 48 h after treatment in MEF (n=2, each condition was done in triplicate and average values are reported). **P<0.01

FIG. 10C shows that in Cyclin E1/E2 KO MEF cells, TAT-CBS is not able to produce an anti-proliferative effect 48 h after treatment. Proliferation values are similar to untreated cells and negative controls (n=2, each condition was done in triplicate and average values are reported).

### DETAILED DESCRIPTION OF THE INVENTION

The invention relates to isolated peptides useful in treatment of cancer by reducing cancer cell proliferation. The peptides are also useful in treatment of vascular disease by reducing smooth muscle cell proliferation. The peptides are useful because they disrupt calmodulin binding to cyclin E protein in order to inhibit CDK2 activity.

In particular, the invention relates to amino acid sequences, termed ‘CBS’-Calmodulin Binding Sequence, that (i) inhibit the binding of CaM to cyclin E, (ii) abrogate Ca2+-sensitive cyclin E/CDK2 activity, (iii) block G1 to S cell cycle progression of vascular smooth muscle cells (“VSMC”) and (iv) reduce their rate of proliferation in vitro. The CBS peptides (v) inhibit the known ‘activating’ phosphorylation of CDK2 on Thr160 (65% inhibition) by selectively inhibiting CaM-cyclin E interactions. The effect is (vi) selective because the binding of CaM to another target protein, calcineurin, was not altered by the CBS peptide, and (vii) both the Ca2+-sensitive CDK2 activity and (viii) proliferation of mouse embryonic fibroblasts deficient in cyclins E (cyclin E1/E2 double knock out mice are embryonic lethal) were not inhibited by the CBS peptide. Finally, (ix) the anti-proliferative effect of the CBS peptides are similar between VSMC (IC50=8.89±1.24 and several human cancer cell lines, such as HeLa (cervical cancer cells), Saos-2 (osteosarcoma), and A549 (lung cancer), and (x) occurs in the absence of cytotoxicity. CBS peptides inhibit (a) cyclin E-specific, Ca2+/CaM-dependent, CDK2 activity, and (b) Ca2+-sensitive cell cycle progression and cell proliferation with high target and mechanistic specificities respectively in both VSMC and human cancer cell lines in vitro. Inhibitors of CaM-cyclin E interactions are therefore useful as therapeutics for diseases characterized by uncontrolled cell proliferation.

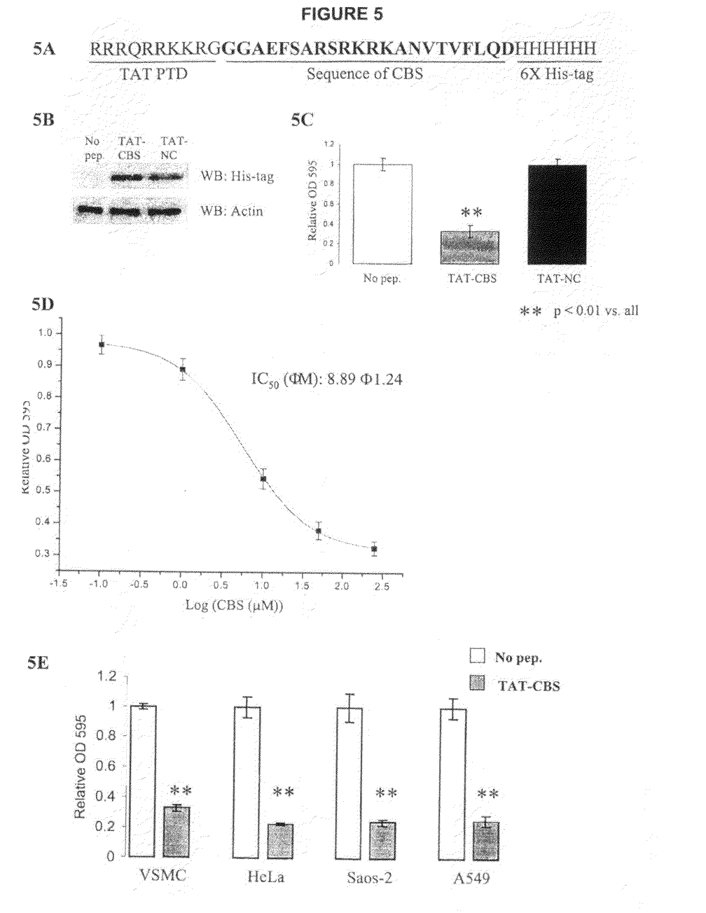

The peptides of the invention have “CBS activity” which can be readily assessed with an assay measuring cell proliferation levels, such as an MIT assay. If a peptide of the invention reduces VSMC or cancer cell proliferation in a statistically significant manner, then it has CBS activity. For example, a peptide having CBS activity optionally reduces proliferation activity by providing at least 50% or at least 70% inhibition effect on the proliferation of VSMC and the cancer cell lines tested in FIG. 5E.

The isolated CBS peptides are useful for treating a number of diseases. In one embodiment, the invention includes methods of reducing cell proliferation caused by cyclin E-specific calcium/calmodulin-dependent CDK2 activity in a cell, by administering a CBS peptide to the cell.

The invention also includes methods of treating cancer in a mammal by administering a CBS peptide to the mammal. Examples of cancer include cervical cancer, osteosarcoma cancer or lung cancer.

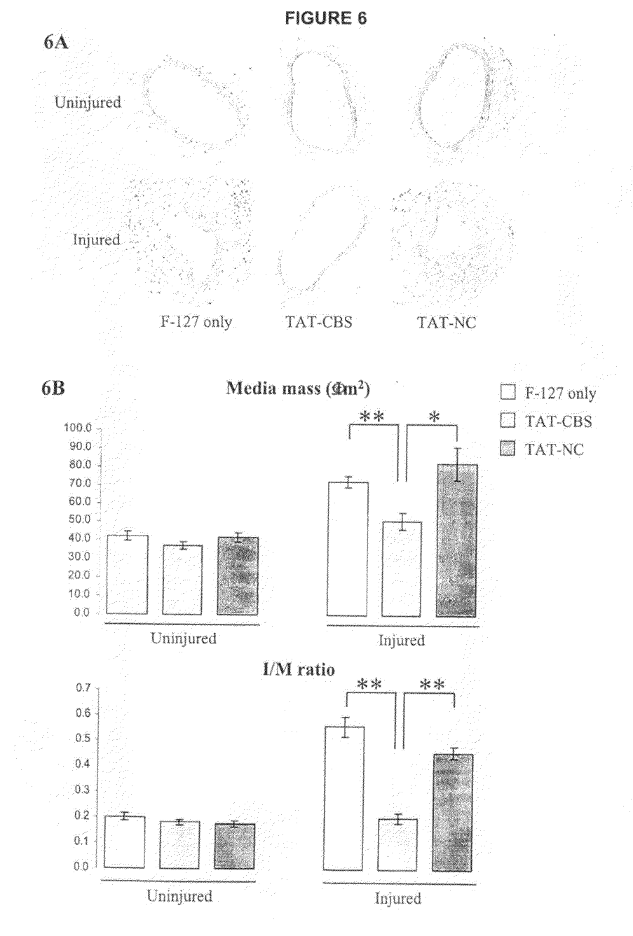

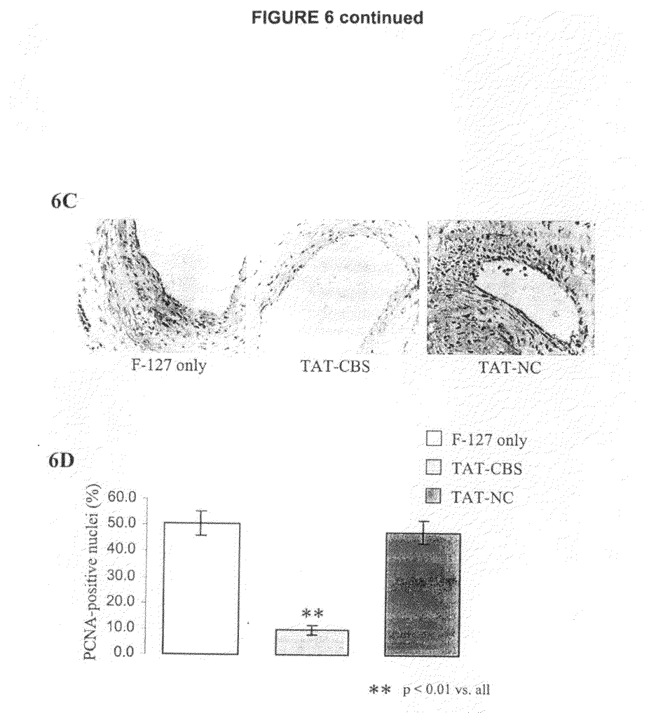

In another embodiment, a CBS peptide is administered to a mammal in a method of reducing proliferation of vascular smooth muscle cells. This effect has been demonstrated in vivo in arteries. Histological examination, morphometry, and immunohistochemical staining for the proliferating cell nuclear antigen (PCNA) of arteries harvested 2 weeks after injury by wire denudation revealed that TAT-CBS(SEQ ID NO:2)-His treatment significantly decreased the vascular smooth muscle cell proliferative response to injury as manifested by reduced medial mass, intima:media ratios, and PCNA-positive nuclei in TAT-CBS(SEQ ID NO:2)-His- vs. F-127 only- and TAT-NC-His-treated groups (FIG. 6)

In another embodiment, the method involves administration of a CBS peptide for treatment of a vaso-occlusive disorder.

The invention also includes an isolated nucleic acid encoding a CBS peptide of the invention. The invention further includes an isolated antibody selectively binding a CBS peptide.

### Peptides of the Invention

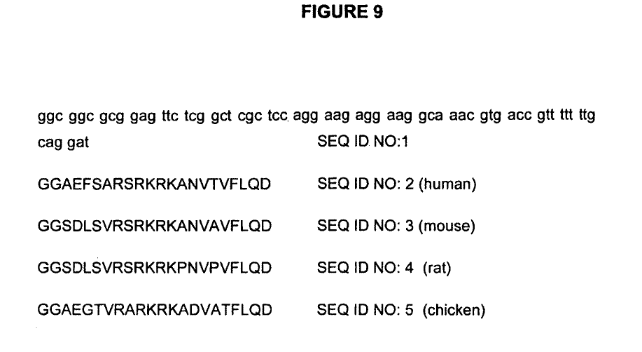

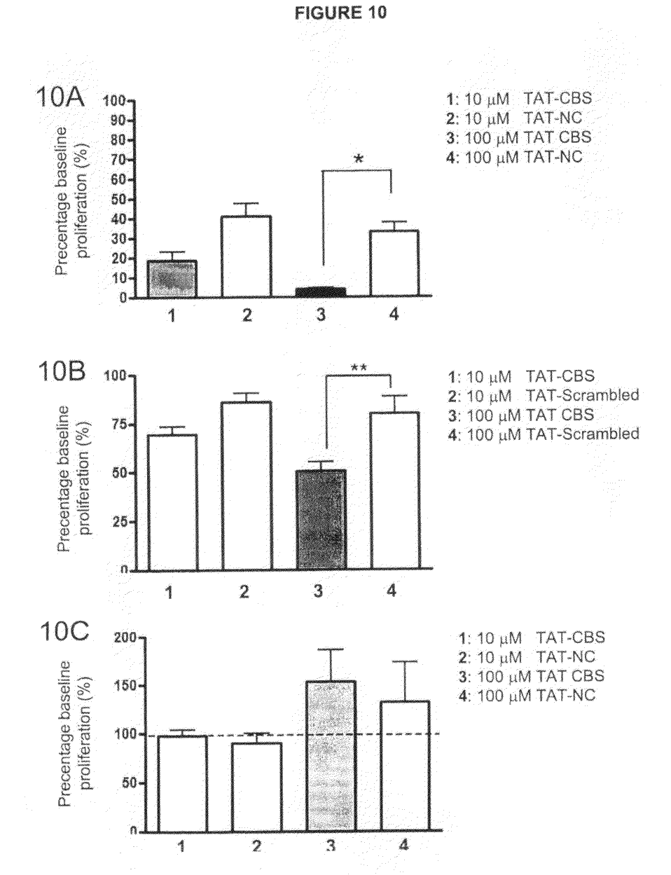

Isolated peptides comprising all or part of any of SEQ ID NO:2 and having cell proliferation inhibition activity are examples of useful peptides (FIG. 9). SEQ ID NO:2 corresponds to the amino acid sequence for the calmodulin (CaM) binding region of human cyclin E1. In alternate embodiments, the invention includes isolated peptides based on homologous 22 amino acid sequences or fragments thereof from other animals, such as mammals and birds. For example, FIG. 9 shows 22 amino acid sequences from rat, mouse and chicken that have CBS activity (SEQ ID NOS:3 to 5). The isolated peptides of the invention are typically 50 amino acids or less, optionally less than: 35, 30, 25, 20, 15, 10. 9, 8, 7 or 6 amino acids long.

In other embodiments, the inventors have shown that peptides as small as 5 amino acids of one of the foregoing sequences have CBS activity and are useful to treat cancer and vascular disease, for example:

### Human: VTVFL

### Mouse: VAVFL

### Rat: VPVFL

### Chicken: VATFL

Optionally, the isolated peptide comprises at least: 5, 6, 7, 8, 9, 10, 11, 15 or 18 amino acids of SEQ ID NO:2-5. Optionally, the peptide comprises a fragment of 5-10, 10-15, 15-20 or 20-21 amino acids of a peptide of SEQ ID NO:2-5 (FIG. 9), wherein the peptide reduces cell proliferation. Examples of useful fragments based on SEQ ID NO:2 include amino acid sequences selected from the group consisting of (amino acids 2-21 of SEQ ID NO:2, amino acids 3-20 of SEQ ID NO: 2, amino acids 4-19 of SEQ ID NO: 2, amino acids 5-18 of SEQ ID NO: 2, amino acids 6-17 of SEQ ID NO: 2, amino acids 7-16 of SEQ ID NO: 2, amino acids 8-15 of SEQ ID NO: 2, amino acids 9-14 of SEQ ID NO: 2, amino acids 12-20 of SEQ ID NO: 2, amino acids 14-20 of SEQ ID NO: 2, amino acids 16-20 of SEQ ID NO: 2).

Particularly useful fragments include peptides having a sequence V-X1-X2-F-L, wherein X1 is optionally a hydrophobic amino acid or a neutral amino acid (eg. selected from the group of V, I, L, F, W, C, A, Y, H, T, S, P, G, R or K), typically T, A, P and wherein X2 is optionally a hydrophobic amino acid or a neutral amino acid (e.g. selected from the group of V, I, L, F, W, C, A, Y, H, T, S, P, G, R or K), typically V or T.

Longer peptide fragments including these 5 mer sequences are also useful, for example, peptides including 5, 6, 7, 8, 9, 10, 11, 15, 18 or 22 amino acids (such as peptides based on SEQ ID NO:2 shown in FIG. 9).

Typically, fragments will comprise three consecutive hydrophobic residues located within 5 amino acids of the C-terminus, such as VFL of SEQ ID NO:2. Optionally, at least one of the hydrophobic residues comprises a leucine residue (see, for example, the location of a leucine in fragments of SEQ ID NO:2).

The peptides of the invention optionally also include analogs of the aforementioned peptides. Analogs of the protein of the invention optionally include, but are not limited to an amino acid sequence containing one or more amino acid substitutions, insertions, deletions and/or mutations. Amino acid substitutions may be of a conserved or non-conserved nature. Conserved amino acid substitutions involve replacing one or more amino acids of the peptides of the invention with amino acids of similar charge, size, and/or hydrophobicity characteristics. When only conserved substitutions are made, the resulting analog should be functionally equivalent. Non-conserved substitutions involve replacing one or more amino acids of the amino acid sequence with one or more amino acids which possess dissimilar charge, size, and/or hydrophobicity characteristics.

One or more amino acid insertions are optionally introduced into the amino acid sequences of the invention. Amino acid insertions consist of single amino acid residues or sequential amino acids ranging for example from 2 to 15 amino acids in length.

Deletions consist of the removal of one or more amino acids, or discrete portions from the amino acid sequence of the peptide. The deleted amino acids may or may not be contiguous.

The peptides of the invention are readily prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

Analogs of a protein of the invention are optionally prepared by introducing mutations in the nucleotide sequence encoding the peptide. Mutations in nucleotide sequences constructed for expression of analogs of a protein of the invention preserve the reading frame of the coding sequences. Furthermore, the mutations will preferably not create complementary regions that could hybridize to produce secondary mRNA structures such as loops or hairpins, which could adversely affect translation of the mRNA.

Mutations are optionally introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures are employed to provide an altered gene having particular codons altered according to the substitution, deletion, or insertion required. Deletion or truncation of a peptide of the invention is also readily achieved by utilizing convenient restriction endonuclease sites adjacent to the desired deletion. Subsequent to restriction, overhangs may be filled in, and the DNA religated. Exemplary methods of making the alterations set forth above are disclosed by Sambrook et al (Sambrook J et al. 2000. Molecular Cloning: A Laboratory Manual (Third Edition), Cold Spring Harbor Laboratory Press).

In addition, analogs of a protein of the invention are readily prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). The peptides of the invention also include peptides having sequence identity to the CBS peptide, mutated CBS peptides and/or truncations thereof as described herein. Such peptides have amino acid sequences that correspond to nucleic acid sequences that hybridize under stringent hybridization conditions (see discussion of stringent hybridization conditions herein) with a probe used to obtain a peptide of the invention. Peptides having sequence identity will often have the regions which are characteristic of the protein.

Peptides preferably have an amino acid sequence with at least 60%, 70%, 80%, 90%, 95%, 96%, 97%, 98%, 99%, preferably 80-95% or more identity with one or more of SEQ ID NOS:2-5 of FIG. 9 or a fragment thereof, wherein the peptide reduces cell proliferation. Sequence identity is typically assessed by the BLAST version 2.1 program advanced search (parameters as above; Altschul, S. F., Gish, W., Miller, W., Myers, E. W. & Lipman, D. J. (1990) “Basic local alignment search tool.” J. Mol. Biol. 215:403_{—}410). BLAST is a series of programs that are available online through the U.S. National Center for Biotechnology Information (National Library of Medicine Building 38A Bethesda, Md. 20894) The advanced Blast search is set to default parameters. References for the Blast Programs include: Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L., Tatusov, R. L. & Zhang, J. (1996) “Applications of network BLAST server” Meth. Enzymol. 266:131-141; Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); Zhang, J. & Madden, T. L. (1997) “PowerBLAST: A new network BLAST application for interactive or automated sequence analysis and annotation.” Genome Res. 7:649-656).

The present invention also includes a protein of the invention conjugated with a selected protein, or a selectable marker protein to produce fusion proteins. The peptide optionally further comprises a TAT linker amino acid sequence comprising all or part of RRRQRRKKRG that increases cellular uptake. Other transduction domains would also be useful to increase cellular uptake having TAT activity of increasing the uptake of peptide into a cell compared to a peptide not having a TAT linker.

For example, the cDNA sequence for a CBS peptide can be inserted into a vector that contains a nucleotide sequence encoding another peptide (e.g. GST-glutathione succinyl transferase). The fusion protein is expressed and recovered from prokaryotic (e.g. bacterial or baculovirus) or eukaryotic cells. The fusion protein can then be purified by affinity chromatography based upon the fusion vector sequence and the CBS peptide obtained by enzymatic cleavage of the fusion protein.

An alternative method of producing the protein is by using a poly-histidine tag. The cDNA sequence is designed to have a poly-histidine tag on the C-terminal end (see in FIG. 5 a His tag at C-terminus). The protein is expressed in prokaryotic or eukaryotic cells, and then easily isolated using a nickel-affinity column. The polyhistidine (usually 6 histidines) adsorbs strongly to the nickel attached to the affinity column while nothing else binds strongly. The ‘his-tagged’ peptide is isolated by washing the column with imidazole.

### Nucleic Acids

The invention includes nucleic acids encoding the CBS peptides of the invention, such as SEQ ID NOS:2-5, or fragments thereof described herein. For example, the invention includes the nucleic acid sequence shown in FIG. 9 SEQ ID NO:1 which encodes SEQ ID NO:2. The invention also includes nucleic acid fragments of SEQ ID NO:1 that encode fragments SEQ ID NO:2 described herein. The proteins of the invention (including truncations, analogs, etc.) are optionally prepared using recombinant DNA methods. Accordingly, nucleic acid molecules of the present invention having a sequence that encodes a peptide of the invention are isolated using known technologies and are incorporated according to procedures known in the art into an appropriate expression vector that ensures good expression of the peptide. The cDNA is preferably obtained by Reverse Transcriptase-Polymerase Chain Reaction (RT-PCR). The technology comes as a kit form. One isolates the messenger RNA that encodes the peptide and then uses reverse transcriptase to convert all messengers in an extract of tissue to cDNA copies. One then amplifies the cloned DNA by standard PCR using a primer synthesized to match a segment of the peptide. The RACE technique is useful to obtain the full mRNA transcript since it codes for a series of peptides that are then cleaved after a bigger protein containing all of them is synthesized. Expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule of the invention and regulatory sequences, selected on the basis of the host cells to be used for expression, which are operatively linked to the nucleic acid molecule. “Operatively linked” means that the nucleic acid is linked to regulatory sequences in a manner that allows expression of the nucleic acid.

The invention also includes nucleic acid sequences with at least 60%, 70%, 75% identity to SEQ ID NO:1.

The invention includes nucleic acid molecules with mutations that cause an amino acid change in a portion of the peptide not involved in providing CBS activity or an amino acid change in a portion of the peptide involved in providing CBS activity so that the mutation increases or decreases the activity of the peptide. The invention includes modified nucleic acid molecules with a sequence identity at least: 60%, 70%, 80% or 90% more preferably at least about 95%, to SEQ ID NO:1 (or a partial sequence thereof). Optionally, 1, 2, 3, 4, 5, 6 to 10 or 10 to 25 nucleotides are modified. Identity is calculated according to methods known in the art. Sequence identity is optionally assessed by the BLAST program (default parameters; references as above).

Other functional equivalent forms of SEQ ID NO:1 encoding peptides with CBS activity are readily identified using conventional DNA-DNA or DNA-RNA hybridization techniques. The present invention also includes nucleic acid molecules that hybridize to SEQ ID NO:1, and that encode peptides exhibiting CBS activity. Such nucleic acid molecules hybridize to all or a portion of SEQ ID NO:1 or its complement under low, moderate, or high stringency conditions as defined herein (see Sambrook et al. (2000). The portion of the hybridizing nucleic acids is typically at least 15 (e.g. 20, 25, 30 or 50) nucleotides in length. The hybridizing portion of the hybridizing nucleic acid is at least: 60%, 70%, 80%, 90%, 95% identical to SEQ ID NO:1 or a portion thereof, or its complement. Hybridizing nucleic acids of the type described herein can be used, for example, as a probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions optionally involve hybridizing at about: 1×SSC, 0.1% SDS at 50° C. for 15 min. High stringency conditions optionally are: 0.1×SSC, 0.1% SDS at 65° C. for 15 min. Moderate stringency is optionally about 1×SSC 0.1% SDS at 60 degrees Celsius for 15 min. The parameters of salt concentration and temperature can be varied to achieve the optimal level of identity between the probe and the target nucleic acid.

The invention therefore includes a recombinant expression vector of the invention containing a nucleic acid molecule of the invention, or a fragment thereof, and the necessary regulatory sequences for the transcription and translation of the inserted peptide-sequence. Suitable regulatory sequences are optionally derived from a variety of sources, including bacterial, fungal, or viral genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native compound and/or its flanking regions.

The invention further provides a recombinant expression vector comprising a DNA nucleic acid molecule of the invention cloned into the expression vector in an antisense orientation. These vectors are useful experimental systems to study the peptides of the invention or its variants or to test antidotes. The peptides may or may not be toxic to the host cells. They are also useful to produce large amounts of the peptide. The vectors are particularly useful because insect-specific biological delivery agents (e.g. viruses) will provide immobilizing agents for specifically targeted insects. Viruses targeted against a specific insect pest are engineered to contain the gene fragment coding for the CBS peptide along with expression regulatory sequences. The virus would target an insect species and then, during reproducing itself, also produce the peptide. Regulatory sequences operatively linked to the antisense nucleic acid can be chosen which direct the continuous expression of the antisense RNA molecule.

The recombinant expression vectors of the invention optionally also contain a selectable marker gene that facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors optionally also contain genes which encode a fusion moiety which provides increased expression of the recombinant protein; increased solubility of the recombinant protein; and aid in the purification of a target recombinant protein by acting as a ligand in affinity purification. For example, a proteolytic cleavage site may be added to the target recombinant protein to allow separation of the recombinant protein from the fusion moiety subsequent to purification of the fusion protein.

Recombinant expression vectors are optionally introduced into host cells to produce a transformed host cell. These cells are useful experimental systems. Accordingly, the invention includes a host cell comprising a recombinant expression vector of the invention. The term “transformed host cell” is intended to include prokaryotic and eukaryotic cells which have been transformed or transfected with a recombinant expression vector of the invention. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al.

Suitable host cells include a wide variety of prokaryotic and eukaryotic host cells. For example, the peptides of the invention may be expressed in bacterial cells such as E. coli, Pseudomonas, Bacillus subtillus, insect cells (using baculovirus), yeast cells or mammalian cells. Other suitable host cells can be found in Goeddel, Gene Expression Technology: Methods in Enzymology 185, Academic Press, San Diego, Calif. (1991).

As an example, to produce peptides recombinantly, for example, E. coli can be used using the T7 RNA polymerase/promoter system using two plasmids or by labeling of plasmid-encoded proteins, or by expression by infection with M13 Phage mGPI-2. E. coli vectors can also be used with Phage lambda regulatory sequences, by fusion protein vectors (e.g. lacZ and trpE), by maltose-binding protein fusions, and by glutathione-S-transferase fusion proteins.

Alternatively, a peptide can be expressed in insect cells using baculoviral vectors, or in mammalian cells using vaccinia virus. For expression in mammalian cells, the cDNA sequence may be ligated to heterologous promoters and introduced into cells, such as COS cells to achieve transient or long-term expression. The stable integration of the chimeric gene construct may be maintained in mammalian cells by biochemical selection, such as neomycin and mycophoenolic acid.

The CBS DNA sequence can be altered using procedures such as restriction enzyme digestion, fill-in with DNA polymerase, deletion by exonuclease, extension by terminal deoxynucleotide transferase, ligation of synthetic or cloned DNA sequences, site-directed sequence alteration with the use of specific oligonucleotides together with PCR. For example, one to five or five to ten amino acids or more may be removed or mutated.

The cDNA sequence or portions thereof, or a mini gene consisting of a cDNA with an intron and its own promoter, is introduced into eukaryotic expression vectors by conventional techniques. These vectors permit the transcription of the cDNA in eukaryotic cells by providing regulatory sequences that initiate and enhance the transcription of the cDNA and ensure its proper splicing and polyadenylation. Different promoters within vectors have different activities which alters the level of expression of the cDNA. In addition, certain promoters can also modulate function such as the glucocorticoid-responsive promoter from the mouse mammary tumor virus.

Some of the vectors listed contain selectable markers or neo bacterial genes that permit isolation of cells by chemical selection. Stable long-term vectors can be maintained in cells as episomal, freely replicating entities by using regulatory elements of viruses. Cell lines can also be produced which have integrated the vector into the genomic DNA. In this manner, the gene product is produced on a continuous basis.

Vectors are introduced into recipient cells by various methods including calcium phosphate, strontium phosphate, electroporation, lipofection, DEAE dextran, microinjection, or by protoplast fusion. Alternatively, the cDNA can be introduced by infection using viral vectors.

CBS peptides may also be isolated from cells or tissues, including mammalian cells or tissues, in which the peptide is normally expressed, for example, by isolating cyclin E1 and cleaving it to a peptide of 22 amino acids or less.

The protein is readily be purified by conventional purification methods known to those in the art, such as chromatography methods, high performance liquid chromatography methods or precipitation.

For example, an anti-CBS peptide antibody (as described below) may be used to isolate a CBS peptide, which is then purified by standard methods.

### Peptide Mimetics

The present invention also includes peptide mimetics of CBS peptides. “Peptide mimetics” are structures which serve as substitutes for peptides in interactions between molecules (See Morgan et al (1989), Ann. Reports Med. Chem. 24:243-252 for a review). Peptide mimetics include synthetic structures which optionally contain amino acids and/or peptide bonds but retain the structural and functional features of a peptide, or enhancer or inhibitor of the invention. Peptide mimetics also include peptoids and oligopeptoids (Simon et al (1972) Proc. Natl. Acad, Sci USA 89:9367).

Peptide mimetics are optionally designed based on information obtained by systematic replacement of L-amino acids by D-amino acids, replacement of side chains with groups having different electronic properties, and by systematic replacement of peptide bonds with amide bonds. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

### Therapeutic Methods

The invention also includes methods of reducing cell proliferation caused by cyclin E specific calcium/calmodulin dependent CDK2 activity in a cell, by administering a CBS peptide to the cell.

The invention also includes methods of treating cancer in a mammal by administering a CBS peptide to the mammal. The cancer is typically of a type that undergoes undergo calcium sensitive cyclin E protein mediated cell proliferation. Examples of cancer cells include a cervical cancer cell, an osteosarcoma cancer cell or a lung cancer cell.

The isolated peptides also provide a novel method of cancer chemotherapy. The peptides are useful as an adjuvant in combination with another chemotherapeutic agent in the treatment of cancer, for example aggressive, rapidly proliferating cancer. The ability of a CBS peptide to selectively inhibit proliferation of several cancer cell lines without inducing cytotoxicity stands in contrast to conventional cell cycle inhibitors. The isolated peptides selectively block CaM-cyclin E interactions, and do not interfere with other Ca2+/CaM-dependent pathways. The peptides do not block low basal levels of CDK2 activity, but only reduce Ca2+-sensitive enhancement of CDK2 activity at the G1 to S cell cycle phase transition, a feature that restrict its effects to rapidly proliferating cells. The peptides provide a safer product for both the prevention and treatment of and cancers.

In another embodiment, a CBS peptide is administered to a mammal in a method of reducing proliferation of vascular smooth muscle cells. The peptide is useful because such vascular smooth muscle cells typically undergo calcium sensitive cyclin E protein mediated cell proliferation.

In another embodiment, the method involves administration of a CBS peptide for treatment of a vaso-occlusive or aneurysm disorder. The vaso-occlusive disorder is typically restenosis, Burger syndrome, atherosclerosis, scleroderma, Raynauds disease, hypertension, pulmonary hypertension or post-vascular surgery smooth muscle cell proliferation. The aneurysm disorder is typically aortic aneurysm, post-angioplasty aneurysm, or post-vascular surgery aneurysm.

In another embodiment, the method involves administration of a CBS peptide for treatment of a visceral smooth muscle cell disorder. The visceral smooth muscle disorder includes inflammatory bowel disease, bowel strictures, spastic bladder, urinary retention and uterine cramps.

A peptide of the invention is optionally infused (i) intravenously, (ii) at the site of angioplasty or aneurysm repair, (iii) in a bypass graft at the time of surgery, or (iv) gradually eluted from a coated stent or other drug-delivery vehicle or device. The peptide prevents restenosis in patients undergoing revascularization. The invention also includes a stent comprising a peptide or pharmaceutical composition of the invention.

The atherosclerosis is typically coronary artery disease, peripheral artery disease or cerebrovascular disease. In one embodiment, the hypertension is caused by smooth muscle cell proliferation. In another embodiment, smooth muscle cell proliferative disorders such as restenosis or aneurysm can occur after vascular surgery. The vascular surgery is optionally selected from the group consisting of coronary angioplasty, coronary stent placement, coronary by-pass surgery, aortic aneurysm repair, aortic or peripheral stent placement, vascular grafting, thrombectomy, vascular angioplasty, and vascular stenting. The peptide or pharmaceutical composition is optionally formulated in a stent. Smooth muscle cell proliferation is caused by vascular injury in certain embodiments.

Accordingly, in one embodiment, the present invention provides a method of reducing cell proliferation or treating one of the aforementioned diseases comprising administering an effective amount of a CBS peptide such as all or part of any one of SEQ ID NOS:2-5, or the other compounds described in this application, to an animal in need thereof. The present invention also provides a use of an effective amount of a CBS peptide to reduce cell proliferation or treat one of the aforementioned diseases. The present invention further provides a use of an effective amount of a CBS peptide in the manufacture of a medicament for reducing cell proliferation or treating one of the aforementioned diseases.

The phrase “reduce cell proliferation” means that the substance results in a decrease in proliferation as compared to a proliferation in the absence of the substance.

### Pharmaceutical Compositions

The isolated polypeptides are optionally purified to at least 95%, 96% or 97%. The polypeptides are also optionally pharmaceutical grade purity (eg. for amino acids, this optionally means in excess of 99% purity, having a uniform crystalline structure, and white in color. The CBS peptides or nucleic acids encoding the CBS peptides, for example, the SEQ ID NO:2 shown in FIG. 9 and fragments thereof, are optionally formulated into a pharmaceutical composition for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including mammals, such as humans.

Administration of an effective amount of pharmaceutical compositions of the present invention provides a dosage for a period of time necessary to achieve the desired result. For example, an effective amount of a substance may vary according to factors such as the disease state, age, sex, and weight of the individual, and the ability of the substance to elicit a desired response in the individual. Dosage regimes are readily adjusted to provide the optimum therapeutic response. For example, several divided doses may be administered daily or the dose may be proportionally reduced as indicated by the exigencies of the therapeutic situation.

The polypeptide of the invention is typically combined with other components such as a carrier in a composition such as a pharmaceutical composition. The compositions are useful when administered in methods of medical treatment, prevention, or diagnosis of a disease, disorder or abnormal physical state.

The pharmaceutical compositions are readily administered to humans or other animals by a variety of methods including, but not restricted to topical administration, oral administration, aerosol administration, intratracheal instillation, intraperitoneal injection, injection into the cerebrospinal fluid, intravenous injection and subcutaneous injection. A stent is optionally used to administer the peptides. Dosages to be administered depend on patient needs, on the desired effect and on the chosen route of administration. Nucleic acid molecules and polypeptides may be introduced into cells using in vivo delivery vehicles such as liposomes. They may also be introduced into these cells using physical techniques such as microinjection and electroporation or chemical methods such as coprecipitation or using liposomes.

The pharmaceutical compositions are prepared by known methods for the preparation of pharmaceutically acceptable compositions which can be administered to patients, and such that an effective quantity of the nucleic acid molecule or polypeptide is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA) or Handbook of Pharmaceutical Additives (compiled by Michael and Irene Ash, Gower Publishing Limited, Aldershot, England (1995). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456.

On this basis, the pharmaceutical compositions optionally include an active compound or substance, such as a peptide or nucleic acid molecule, in association with one or more pharmaceutically acceptable vehicles or diluents, and contained in buffered solutions with a suitable pH and iso-osmotic with the physiological fluids. The methods of combining the active molecules with the vehicles or combining them with diluents is well known to those skilled in the art. The composition optionally includes a targeting agent for the transport of the active compound to specified sites within tissue.

### Preparation of Antibodies

Antibodies to the peptide are useful to purify CBS peptides (eg. by contacting CBS peptide with antibody bound to a resin in column chromatography). Antibodies are also useful to monitor CBS peptides in experiments. Antibodies to the 22 mer are useful to monitor CBS peptide delivery and location in animal and humans, and are also useful as an-‘antidote’ to prevent or reduce unexpected toxicity or overdosage of the therapeutic based on the 22 mer. Any method of labeling the peptide that would report on peptide density/location would be useful (e.g. radioactively labeled peptide or fluorescently tagged peptide). Such experiments test how the CBS peptide works in other tissues or animals or people. Antibodies are typically generated against epitopes of the sequences such as SEQ ID NOS:2-5. The antibody is optionally labeled with a detectable marker or unlabeled. The antibody is typically a monoclonal antibody or a polyclonal antibody. Such antibodies are employed to screen organisms. The antibodies are also valuable for immuno-purification of polypeptides from crude extracts. For example, one may contact a biological sample with the antibody under conditions allowing the formation of an immunological complex between the antibody and a polypeptide recognized by the antibody and detecting the presence or absence of the immunological complex whereby the presence of the peptide of the invention or a similar peptide is detected in the sample. The invention also includes compositions preferably including the antibody, a medium suitable for the formation of an immunological complex between the antibody and a polypeptide recognized by the antibody and a reagent capable of detecting the immunological complex to ascertain the presence of the peptide of the invention or a similar polypeptide.

To recognize the peptide of the invention, one readily generates antibodies against a unique epitope in the CBS peptide.

Monoclonal and polyclonal antibodies are prepared according to the description in this application and techniques known in the art. For examples of methods of the preparation and uses of monoclonal antibodies, see U.S. Pat. Nos. 5,688,681, 5,688,657, 5,683,693, 5,667,781, 5,665,356, 5,591,628, 5,510,241, 5,503,987, 5,501,988, 5,500,345 and 5,496,705 that are incorporated by reference in their entirety. Examples of the preparation and uses of polyclonal antibodies are disclosed in U.S. Pat. Nos. 5,512,282, 4,828,985, 5,225,331 and 5,124,147 which are incorporated by reference in their entirety.

The term “antibody” includes fragments thereof which also specifically react with a CBS peptide or fragments thereof. Antibodies are fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules include, for example, the antigen binding domain from an antibody of a mouse, rat, or other species, with human constant regions. Conventional methods are useful to make chimeric antibodies containing the immunoglobulin variable region which recognizes the CBS peptide antigens of the invention (See, for example, Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules are made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, such as, but not limited to, single-chain Fv monoclonal antibodies reactive against the peptides of the invention may also be generated by screening expression libraries encoding immunoglobulin genes, or portions thereof, expressed in bacteria with peptides produced from the nucleic acid molecules of CBS peptides. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

The invention also includes methods of using the antibodies, such as in detection of receptors that bind to the peptide of the invention. For example, the invention includes a method for detecting the presence of a peptide of the invention by: a) contacting a sample containing one or more peptides with an antibody of the invention under conditions suitable for the binding of the antibody to peptides with which it is specifically reactive; b) separating unbound peptides from the antibody; and c) detecting antibody which remains bound to one or more of the peptides in the sample.

### Research Tool

The peptides (SEQ ID NO:2-5) and fragments thereof are useful in research protocols to further investigate cell proliferation in VSMC and cancer cells. For example, experiments requiring the cell cycle arrest of VSMC and cancer cells use the peptides and fragments thereof. The invention includes the use of a peptide of the invention to synchronize cells (eg. smooth muscle cells or cancer cells) at the G1 phase, for example, in a method of synchronizing cells at the late G1 phase by administering to the cells a peptide of the invention.

Additional uses include biological investigations into the cellular and molecular physiology of cell cycle and processes dependent on cell cycle progression. The peptides (SEQ ID NO:2-5) and their fragments are useful as positive and negative controls for in vitro and in vivo studies of other agents useful in inhibiting vascular smooth cell proliferation, cell cycle progression, interactions between CaM and cyclin E or other CaM-binding proteins, cyclin E-dependent CDK2 activity, and Ca2+-sensitive CDK2 activity or cell-cycle progression.

### Materials and Methods

Materials. Purified active cyclin E/CDK2 and CaM were obtained from Upstate Biotech (Lake Placid, N.Y.). Calmidazolium, DTT, aprotinin, leupeptin, Thiazolyl Blue Tetrazolium Bromide (MTT), and other chemicals were purchased from Sigma (St. Louis, Mo.). Synthetic peptides were purchased from GenScript Corp. (Piscataway, N.J.). Pluronic F-127 Gel was obtained from BASF (Ludwigshafen, Germany).

Cell culture conditions The isolation and culture of primary mouse aortic VSMC have was performed according to methods previously described (Afroze T, Yang L L, Wang C, Gros R, Kalair W, Hogue A N, Mungrue I N, Zhu Z, Husain M. Calcineurin-independent regulation of plasma membrane Ca2+ ATPase-4 in the vascular smooth muscle cell cycle. Am J Physiol Cell Physiol. July 2003; 285(1):C88-95). VSMCs were grown in DMEM (GIBCO/BRL, Gaithersburg, Md.) supplemented with 10% FBS (Hyclone, Logan, Utah), 50 ng/mL platelet-derived growth factor (PDGF, Sigma) and 1% penicillin-streptomycin (GIBCO/BRL). For serum starvation, cells were grown to 60% to 70% confluence, washed twice with PBS, and cultured in starvation medium (0.25% FBS-supplemented DMEM) for 72 hours to achieve Gₒ arrest. Progression to G₁/S was brought about by stimulation with 10% FBS and 50 ng/mL PDGF for 24 hours. Cells and reagents for primary human aortic VSMC culture were purchased from Cascade Biologics. Human VSMC(C-007-5C) were grown in Medium 231 with Smooth Muscle Growth Supplement (SMGS) S-007-25 and 1% penicillin-streptomycin (GIBCO/BRL). Cyclin E1/E2 double knockout mouse embryonic fibroblasts (MEF) were kindly provided by Dr. P. Sicinski (Harvard Medical School), and maintained in DMEM with 10% FBS (Hyclone) and 1% penicillin-streptomycin (GIBCO/BRL). Gₒ arrest of MEF was achieved by starvation for 48 h in medium lacking FBS. Mouse embryonic fibroblasts (MEF) were maintained for normal culture in 10% FBS-supplemented DMEM. For the starvation of MEF, cells were cultured in starvation medium without FBS for 48 hours to achieve Gₒ arrest. HeLa (human cervical cancer cell), Saos-2 (human osteosarcoma cell) and A549 (human lung cancer cell) were cultured in 10% FBS-supplemented DMEM.

Immunoprecipitation and kinase assays. 5×10⁶ SMC were harvested and re-suspended with 2 ml of lysis buffer (50 mM Tris (pH 7.4), 250 mM NaCl, 5 mM EDTA, 0.1% NP-40, 0.5 mM DTT, 0.1 mM Na₃VO₄, 5 μg/ml leupeptin, 5 μg/ml aprotinin, 2 mM PMSF, and 10% glycerol). After homogenization, cell lysates were clarified by centrifugation at 12×10³ rpm, 4° C. for 30 min, and the supernatant was collected. An aliquot was taken for protein concentration determination using the BCA protein assay kit (Sigma). Cell extracts (200 μg) were then incubated for 2 hr at 4° C. with saturating concentrations of polyclonal antibodies (Abs). Immune complexes were collected by incubation with GammaBind™ G Sepharose resin (Amersham Pharmacia, Piscataway, N.J.) for 1 h at 4° C. The beads were then washed three times with washing buffer (50 mM Tris (pH 7.4) and 1 mM DTT).

For the in vitro kinase assays, 20 μl of kinase reaction mixture (20 mM Tris (pH 7.4), 5 mM MgCl₂, 2.5 mM MnCl₂, 1 mM DTT, 10 μg of C-terminal of human Rb protein (Upstate Biotech.) or 4 μg of histone-H1 (Upstate Biotech.) as substrates, 13 μM ATP, and 2.4 μCi [γ-³²P]ATP (Amersham Pharmacia)) was added to CDK2-, cyclin E-, or cyclin A-immunoprecipitated (IPd) complexes after removing the washing buffer from GammaBind™ G Sepharose resin. Based on the total calcium content of kinase reaction mixtures (as determined by inductively coupled plasma atomic emission spectrometry), amounts of EGTA required to yield final concentrations of free Ca²⁺ ion (0, 100 or 500 nM) were calculated by WinMaxC (http://www.stanford.edu/˜cpatton/winmaxc2.html). After 30 min of incubation at 37° C., reactions were stopped by adding 20 μl of 2×SDS loading buffer and heating at 100° C. for 5 min. Labeled proteins were resolved by 16% SDS PAGE. Phosphorylated Rb or histone-H1 bands were visualized by autoradiography and quantified in a Scintillation Counter LS6500 (Beckman Coulter, Fullerton, Calif.) after being cut from the gel.

Western blot. Between 20-30 μg of cell extracts were resolved by 12% SDS PAGE and transferred to nitrocellulose membranes (Sigma, 0.2 μM pore size). Blots were blocked with 5% nonfat dry milk in TBS-T (10 mM Tris (pH 8.0), 150 mM NaCl, and 0.05% Tween 20) overnight at 4° C. and then incubated for 3 h at 25° C. in TBS-T plus 3% nonfat dry milk containing primary Abs, including anti-CDK2 (Upstate Biotech), -cyclin E (Abcam, Cambridge, Mass.), -phosphorylated CDK2 on Thr¹⁶⁰ (Cell Signaling Technology, Danvers, Mass.), -CaM, -phosphorylated CDK2 on Thr¹⁴/Tyr¹⁵, and -actin (Santa Cruz Biotechnology, Santa Cruz, Calif.). Protein bands were detected with ECL Reagents (GE Healthcare), with horseradish peroxidase-conjugated secondary goat anti-rabbit IgG (Santa Cruz Biotech.). Quantification of band intensity was performed using Quantity One (Bio-Rad, Hercules, Calif.).

MTT colorimetric survival assay. Active mitochondria of living cells can cleave MTT to produce formazan, the amount of which is directly proportional to the number of living cells. Cell survival was assessed (Denizot, 1986). Cells in 96-well plate were washed two times with PBS followed by 4 hr of incubation at 37° C. with 1 mg/ml of MTT (in phenol red free DMEM supplemented with 2 mM of L-glutamine & 1 mM of sodium pyruvate) in a reaction volume of 100 μl. After removing MTT solution, DMSO was added to dissolve formazan crystals. Plate was shaken for 5 min at 55° C. for complete dissolve. Dye absorbance in viable cells was measured at 570 nm with 630 nm as a reference wavelength. All experiments were repeated at least 3 times, and each experimental condition was repeated in at least quadruplicate wells in each experiment.

Peptide Delivery. For treatments with peptides, cells were seeded in 24-well plates, grown to 60% confluence, washed twice with PBS and administered peptide solutions consisting of varying doses of peptide and their appropriate serum-free medium (Medium 231 without SMGS for primary VSMC, DMEM without FBS for MEF) for 1 h at 37° C. Cells were then washed twice with PBS, and immediately given complete medium (Medium 231 containing SMGS or DMEM supplemented with FBS for MEF).

³H-thymidine incorporation proliferation assay. Incorporation of ³H-thymidine during DNA synthesis was used as another marker of cell proliferation. After treatment with peptides, 0.5 μCi of ³H-thymidine (Perkin-Elmer; Cat No: NET027Z) was administered to each well of a 24 w plate. Cells were grown at 37° C. and analyzed at 24, 48 and 72 h after ³H-thymidine exposure. At the time of harvest, cells were washed twice with ice-cold PBS, and incubated with 10% trichloroacetic acid (TCA) on ice for 10 min to precipitate macromolecules. Two more incubations with fresh 10% TCA on ice were carried out for 5 min each. TCA was removed, and precipitates were dissolved by vigorous shaking at room temperature for 5 min with a solution of 0.2M NaOH and 1% SDS. Solubilized contents of wells were removed and added to 2.5 ml of scintillation fluid (ReadySafe™ Cocktail, Beckman Coulter, Product No. 141349). Radioactivity was measured in a scintillation counter (Beckman Coulter, LS 6500).

Lactate dehydrogenase (LDH) assay. To measure the level of cytotoxicity, LDH based in vitro toxicology assay kit (Sigma, Cat. No.: TOX-7) was used (Legrand, 1992). After nucleofection of peptides, 10⁶ cells were grown on 24-well plate in the presence of phenol red free DMEM. 3 days later, 500 μl of cell culture media was collected from each well for assay followed by centrifugation (12,000 rpm, 30 min, at 4° C.) to remove any cell debris completely. Subsequent procedures for enzymatic assay were performed according to manufacture's protocol.

Confocal immunofluorescence. VSMCs harboring FITC-labeled peptides were fixed with 2% paraformaldehyde for 30 min and washed with PBS 3 times for 5 min each. Cells were incubated with 10 μg/ml Hoechst dye in the dark for 15 min at RT. After 5 times of washing with PBS, mounted slides were examined with an Olympus Fluoview 1000 confocal microscope (Olympus America Inc. Melville, N.Y.) and images were obtained with FV10-ASW software.

Nucleofection. For peptides delivery via Nucleofector technology, 1×10⁶ cells were harvested, resuspended in 100 μl of Nucleofector solution (for VSMC: Cat. No. VPI-1004; for MEF: Cat. No.: VPD-1005, both provided by Amaxa GmBH, Köln, Germany) and mixed with 100 nmole (in 10 μl) of each peptide. The cell solution was transferred into a kit-provided cuvette and positioned into a Nucleofector device. Subsequently, the transfection was performed with a single pulse using the preprogrammed nucleofection program (A-33 for VSMC; T-20 for MEF) according to the manufacturer's recommendation. Following nucleofection, the cells were transferred into 96-well plate (2K or 5K cells per well for MTT assay) or T25 culture flasks (for FACS analysis) containing pre-warmed stem cell medium using kit-provided plastic pipettes to prevent cell damage and the cells were cultured at 37° C. in 5% CO₂.

Flow cytometric analysis. 1*10⁶ cells were trypsinized, washed twice in PBS, and finally resuspended in 0.2 ml of PBS. 5 ml of ice cold 70% ethanol was added dropwise while vortexing gently and cells were then kept on ice for 1 hr. After centrifugation at 500×g for 2 min at 4° C., cells were resuspended in 0.5 ml 2N HCl with 0.5% Triton X-100 and incubated at room temperature for 30 min followed by centrifugation. Cells were again resuspended in 1 ml of 5 μg/ml propidium iodide (PI), and 0.5 mg/ml RNase A in PBS and incubated for 2 hrs on ice and in the dark. Stained cells were filtered through 40 μm nylon mesh capped tubes just prior to flow cytometry to remove cell clumps. Cells were counted in a flow cytometer (FACScan, BD Biosciences), and G₀/G₁, S, and G₂/M cell percentages were calculated with Cell Quest software (BD Biosciences).

Injury of mouse carotid arteries (wire denudation) and local administration of CBS peptide. The mice (8-12 weeks, 20-25 g) were anesthetized with i.p. Ketamine (100 mg/kg, MTC Pharmaceuticals, Cambridge, ON, Canada) and Xylazine (10 mg/kg, Bayer Inc., Etobicoke, ON, Canada). Surgical procedures were performed under a dissecting microscope (MZ6 Leica, Heerbrugg, Switzerland). The left carotid artery was exposed via a midline incision on the ventral side of the neck. The bifurcation of the carotid artery was located and two ligatures were placed around the external carotid artery. This vessel was then tied off with the distal ligature. Temporary occlusion of the internal carotid artery was achieved with a vascular clamp. An incision hole is made between the two ligatures of external carotid artery, through which the denudation device is introduced. This device consists of a curved flexible copper wire (0.3 mm diameter) which is passed down to the common carotid artery, and under rotation, passed along this segment four times. The wire was then removed, and the external carotid artery was tied off proximal to the incision hole with the proximal ligature. After injury, common carotid arteries were surrounded by 100 μl of 25% (wt/vol) pluronic F-127 gel with or without peptides (TAT-CBS & TAT-NC, both 250 μM) before closure of the wound. The mice were allowed to recover in a warmed and padded cage.

Tissue processing and analysis. At varying time points (between 2 days and 6 weeks) following surgery, the animals were anesthetized and previously injured common carotid arteries were harvested for pathological analysis. The animals were then euthanized, with either a lethal dose of Avertin (2% 1 ml i.p.) or Sodium Pentobarbital (100 mg/kg) prior to their recovery from this anesthetic. Anesthetized animals were arrested in diastole by 2 M KCL injection in the left ventricle (LV) and perfusion fixed at physiological pressure with 4% buffered paraformaldehyde by the same LV puncture. The carotid arteries collected were post-fixed in 10% formalin in PBS, embedded in paraffin, sectioned (4 μm) and stained with hematoxylin & eosin (H&E). Images of stained sections were acquired on a DMLB microscope (Leica Microsystems, Richmond Hill, ON, Canada) fitted with Photometrics Cool-snap digital camera (Carsen Group, Markham, ON, Canada). Images were analyzed by planimetry using Image J software. The mean luminal and whole areas were obtained by averaging triplicate values at the proximal, middle, and distal thirds of each collected arterial sample. Medial mass was calculated subtracting the luminal from whole area.

Immunostaining. For immunostaining, arterial sections were treated with 3% H₂O₂ and blocked with primary blocking serum for 1 hour. Overnight incubation at 4° C. was done with a rabbit anti-His-Tag polyclonal antibody (1:500) (Cell Signaling Technology). Sections were washed with PBS 3 times, for 5 min each on next day and incubated with a biotinylated secondary antibody (1:250) (Vector Lab Inc. Burlingame, Calif.) for 1 hour. The sections were then washed and treated with strepavidin-peroxidase for 30 min and treated with diaminobenzidine (DAB) for 2-3 min and mounted after counterstaining with hematoxyline.

Statistics. ANOVA, Student's t-test, and Coefficients of Variance tests were employed as appropriate. Unless otherwise detailed with specific P values, statistical significance was defined as P<0.05. Analyses were performed on SPSS v13.0 (Chicago, Ill.).

### EXAMPLES

The following examples illustrate embodiments of the invention and do not limit the scope of the invention.

### Example 1

### Sequence-Specific Activity of CBS

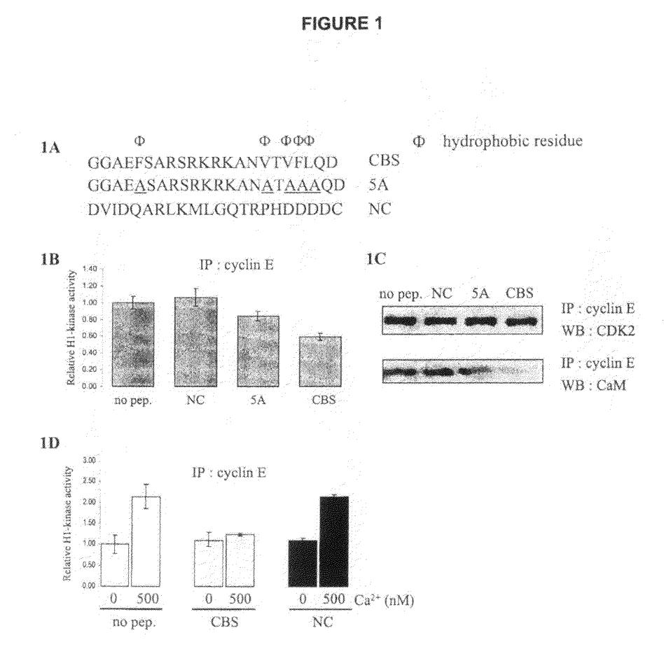

Three distinct synthesized peptides were tested (FIG. 1A). The first was the full 22 mer CBS peptide (SEQ ID NO:2). The second was ‘5A’, which harbors 5 separate alanine substitutions at each of the 5 hydrophobic residues found in SEQ ID NO:2. This enabled the role of normally critical hydrophobic residues in the binding of CaM to target proteins to be shown. A negative control ‘NC’, with the same length, 22 amino acids, as SEQ ID NO:2 was also synthesized. A Histone H1 in vitro kinase assay showed that CBS exhibited 40% inhibition of in vitro cyclin E/CDK2 activity, while NC showed no effect at all. Partial inhibition was observed with 5A, supporting the importance of hydrophobic residues within SEQ ID NO:2 (FIG. 1B). Co-immunoprecipitation analyses showed that cyclin E/CDK2 complex formation was not affected by the SEQ ID NO:2. Rather, the cyclin E-CaM interaction was significantly reduced by the CBS peptide, showing this as the mechanism of diminished cyclin E/CDK2 activity (FIG. 1C). Ca²⁺-sensitive enhancement of cyclin E/CDK2 activity was also abrogated by SEQ ID NO:2, while NC-treatment had no significant effect (FIG. 1D).

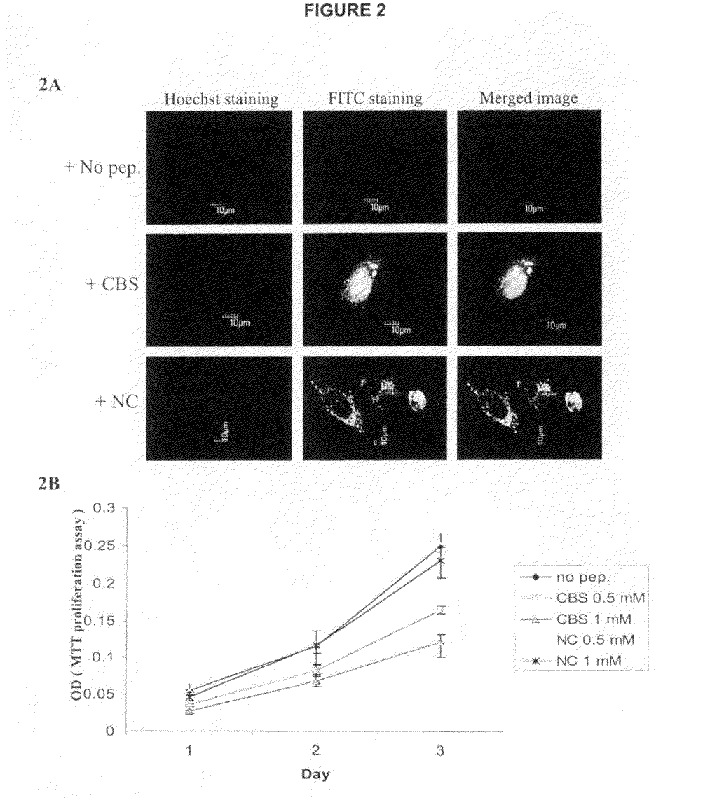

To investigate the effects of SEQ ID NO:2 on the proliferation of VSMC, SEQ ID NO₂ or NC peptides were delivered into primary mouse aortic SMC via nucleofection (i.e. nucleus-targeted electroporation). Confocal microscopy images of a FITC-tagged SEQ ID NO:2 confirmed the delivery of SEQ ID NO:2 into the nuclei of VSMC with an efficiency of ˜40% (FIG. 2A). MTT colorimetric survival assays (proliferation assays) showed that SEQ ID NO:2 delayed and inhibited the proliferation of these VSMC in a dose-dependent manner (FIG. 2B).

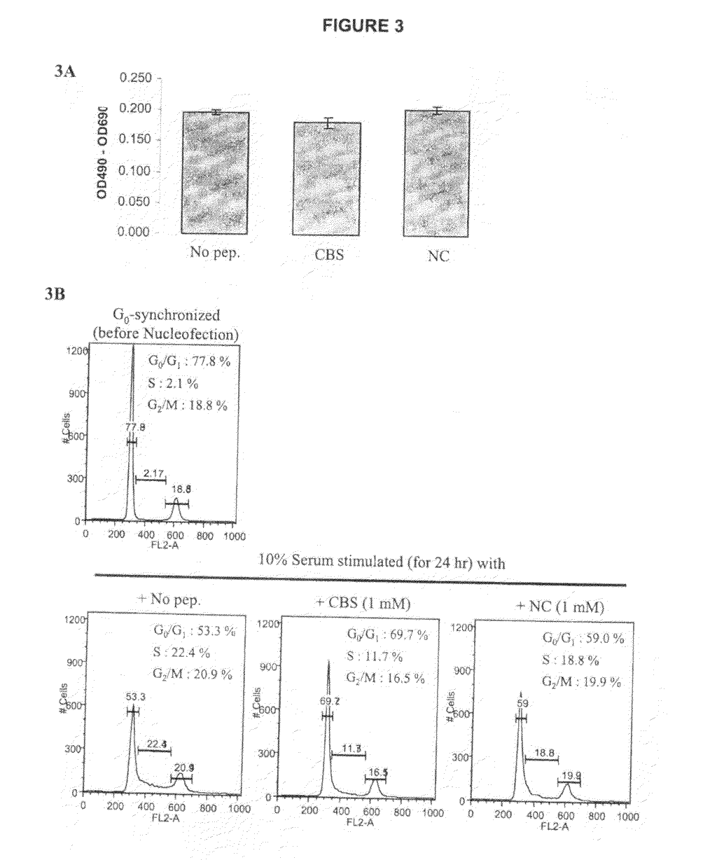

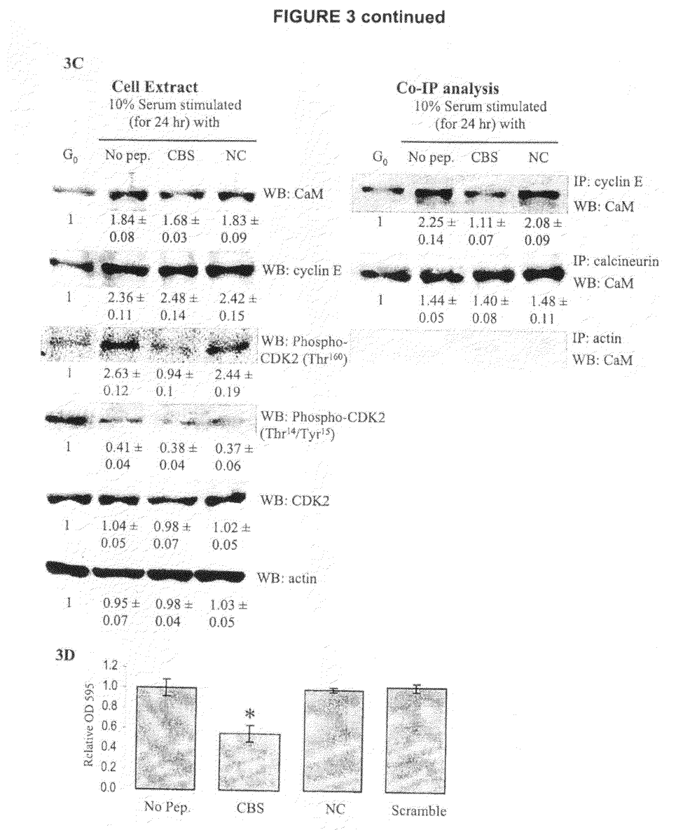

To show the mechanism(s) of CBS peptide-induced proliferation, LDH assays (providing evidence of cytotoxicity) were also performed. These showed that the nucleofection of SEQ ID NO:2 did not cause any increase in cell death (FIG. 3A). Rather, FACS-based analysis of cell cycle progression showed that the effect on cell proliferation was due to G₁ phase cell cycle arrest (FIG. 3B). Western blot and co-immunoprecipitation analyses revealed that SEQ ID NO:2 inhibited CaM-cyclin E interactions and the phosphorylation of CDK2 on Thr¹⁶⁰ (65% inhibition), a known event in the activation of CDK2. SEQ ID NO:2 did not alter the level of inhibitory phosphorylation of CDK2 indicating that the amount of CaM and cyclin E itself was not altered by SEQ ID NO:2, but the interaction between the two proteins was significantly reduced. SEQ ID NO:2 did not alter the binding of CaM to another target protein, calcineurin, showing the high selectivity of SEQ ID NO:2 (FIG. 3C). Proliferation assays with the scrambled (sequence: FAFGRQVNKARSEKALGVSDRT) peptide showed no effect on VSMC (FIG. 3D). This further confirmed that the secondary structure of SEQ ID NO:2 is relevant.

### Example 2

### Cyclin E-Specific Activity of CBS

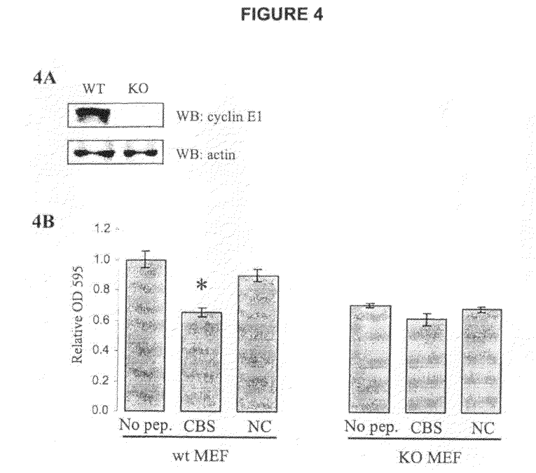

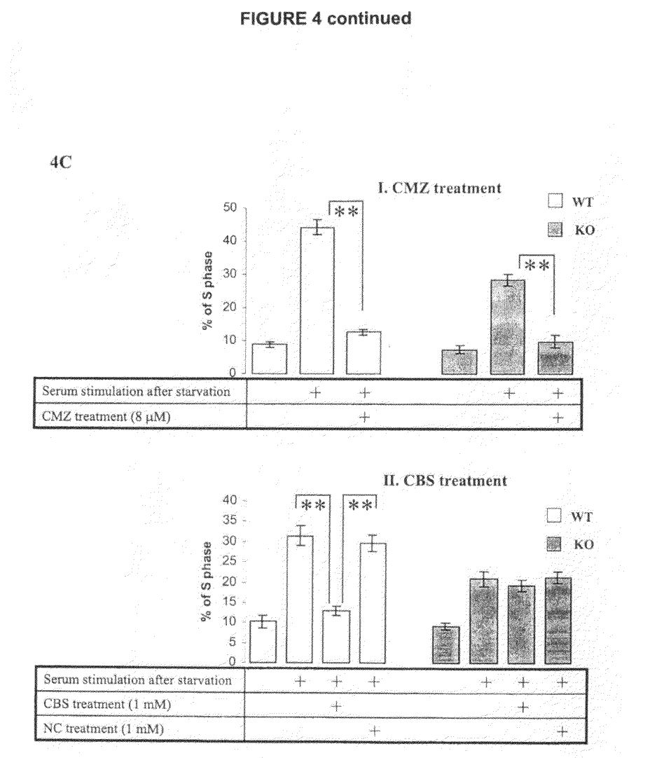

The proliferation of wild-type mouse embryonic fibroblasts (“MEF”) was also inhibited by SEQ ID NO:2, at a level similar to that in VSMC. However, the proliferation of MEF lacking cyclins E1 and E2 (double knockout MEF) was not affected, showing that CBS peptides inhibit the proliferation of VSMC in a cyclin E-specific manner (FIG. 4B). Similarly, cell cycle progression into S phase was inhibited by SEQ ID NO:2 in wild-type MEF, but not in cyclins E1/E2 double knockout MEF. In contrast, calmidazolium (CMZ), a non-selective CaM-inhibitor, did inhibit cell cycle progression of double knockout MEF, possibly via other CaM-dependent pathways (FIG. 4C).

### Example 3

### The TAT Domain Increases the Efficacy of CBS

CBS peptides are useful therapeutics, however, the invention also includes use of modified CBS peptides that have increased efficacy. The TAT protein transduction domain from HIV-1 was fused to the N-terminus of SEQ ID NO:2 peptide to create an alternative method of delivering CBS peptides into animal cells in vivo. This was based on the ability of the TAT domain to deliver a size-independent variety of molecules into the nucleus of cells (Wadia, 2005 #169). A 6×-His tag was also fused to the C-terminus of SEQ ID NO: 2 (FIG. 5A). Western blotting with His-tag antibody following treatment with TAT-SEQ ID NO:2-His or TAT-NC-His to primary VSMC confirmed the delivery of TAT-fused peptides into cells (FIG. 5B). Proliferation assay shows that the anti-proliferative effects of TAT-SEQ ID NO:2-His (FIG. 5C). The TAT-SEQ ID NO:2 peptide reduced the proliferation of VSMC in a dose-dependent manner, with an IC₅₀ value of 8.89±1.24 μM (FIG. 5D). Of note, the maximum level of inhibition was also improved by the TAT-SEQ ID NO:2 fusion as compared to the CBS alone (70% vs. 50%). Finally, the proliferation of three distinct human cancer cell lines—HeLa (cervical cancer), Saos-2 (osteosarcoma), and A549 (lung cancer)—was also inhibited by TAT-SEQ ID NO:2 with a level of inhibition similar to that in VSMC (FIG. 5E).

### Example 4

### In vivo Application of TAT-CBS to Mouse Carotid Artery Injury Model

Histological examination, morphometry, and immunohistochemical staining for the proliferating cell nuclear antigen (PCNA) of arteries harvested 2 weeks after injury revealed that TAT-CBS(SEQ ID NO:2)-His treatment significantly decreased the vascular smooth muscle cell proliferative response to injury as manifested by reduced medial mass, intima:media ratios, and PCNA-positive nuclei in TAT-CBS(SEQ ID NO:2)-His- vs. F-127 only- and TAT-NC-His-treated groups (FIG. 6). The application of TAT-SEQ ID NO:2-His peptides to the site of injury on mouse carotid arteries, induced by wire denudation method, significantly decreased the thickening of arterial media when compared to F-127 only and TAT-NC treatments (FIG. 6A). It has been known that the migration of VSMC from media to intima after its initial proliferation in media is one of the most essential steps in the development of atherosclerosis. Accordingly, total Media Mass in the artery and the ratio of intima to media (I/M ratio) were measured, revealing the anti-proliferative effect of TAT-SEQ ID NO: 2 when compared to F-127 solution only and TAT-NC treatments (FIG. 6B). Proliferating cell nuclear antigen (PCNA) staining confirmed the reduction of cell proliferation by TAT-SEQ ID NO:2 peptide at injured arteries (FIGS. 6C & D).

### Example 5

### Truncated CBS Peptides Reduce Proliferation of Cancer Cells

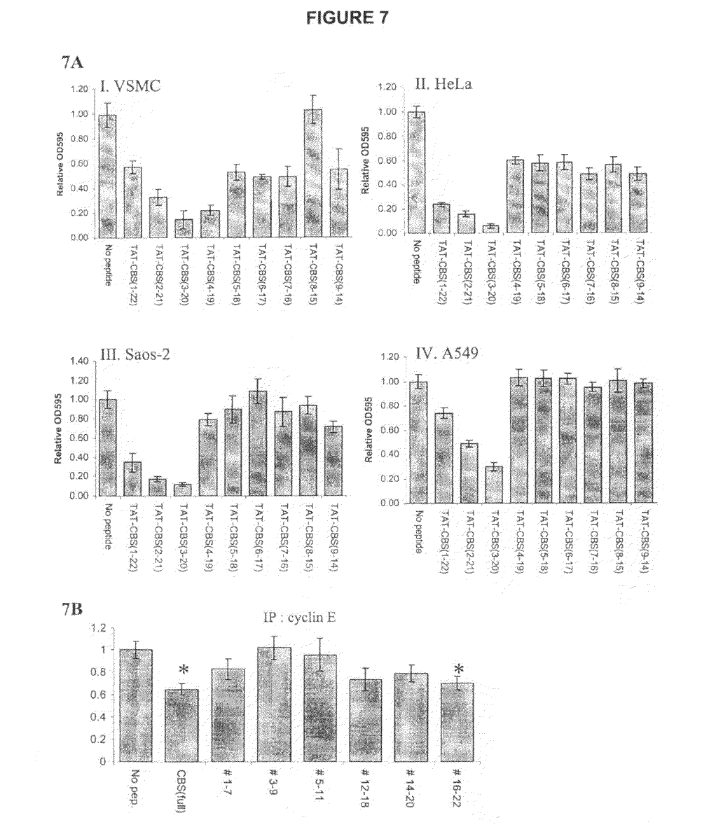

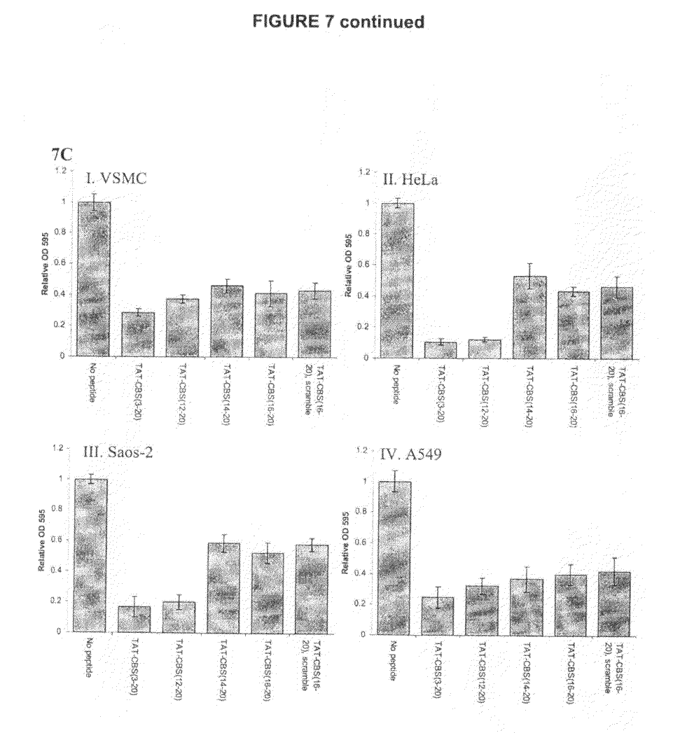

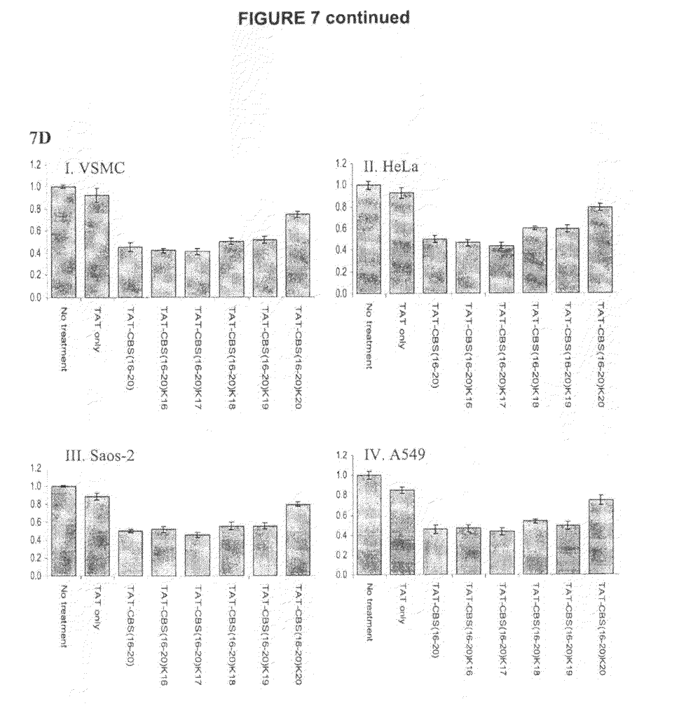

Proliferation assays with a series of truncated SEQ ID NO:2 (CBS) peptides (shown in Table 1) revealed that TAT-SEQ ID NO:2 (#3-20) peptide reduced proliferation of primary VSMC and cancer cells (HeLa, Saos-2 and A549 cells) most powerfully and efficiently, whereas TAT-SEQ ID NO:2 (#4-19) started to lose its proliferative function (FIG. 7A). Since it is generally believed that hydrophobic and basic residues in the general calmodulin binding domain are critical for the binding of CaM and since alanine is generally thought to have little function, the proliferation levels resulting from the truncation from TAT-SEQ ID NO:2 (#3-20) to TAT-SEQ ID NO:2 (#4-19), which removes Alanine and Leucine (from positions 3 and 20, respectively) indicates that Leucine (#20) might be an important residue in SEQ ID NO:2. Histone H1 kinase assay with truncated SEQ ID NO:2 peptides (shown in Table 1B) showed that 7 amino acids (#16-#22) in SEQ ID NO:2 play a critical role in reducing cyclin E/CDK2 activity (FIG. 7B). MTT assays with additional truncated SEQ ID NO:2 peptides (sequences shown at Table 1C) revealed that anti-proliferative effects were present with as little as 5 amino acids from SEQ ID NO: 2 (FIG. 7C). MTT assays with the sequences shown at Table 1D also showed that the TAT domain itself has no anti-proliferative effect and that Leucine (#20) is an important residue in SEQ ID NO:2 (FIG. 7D).

### Example 6

### In Vivo Application of Truncated CBS Peptides to Mouse Carotid Artery Injury Model

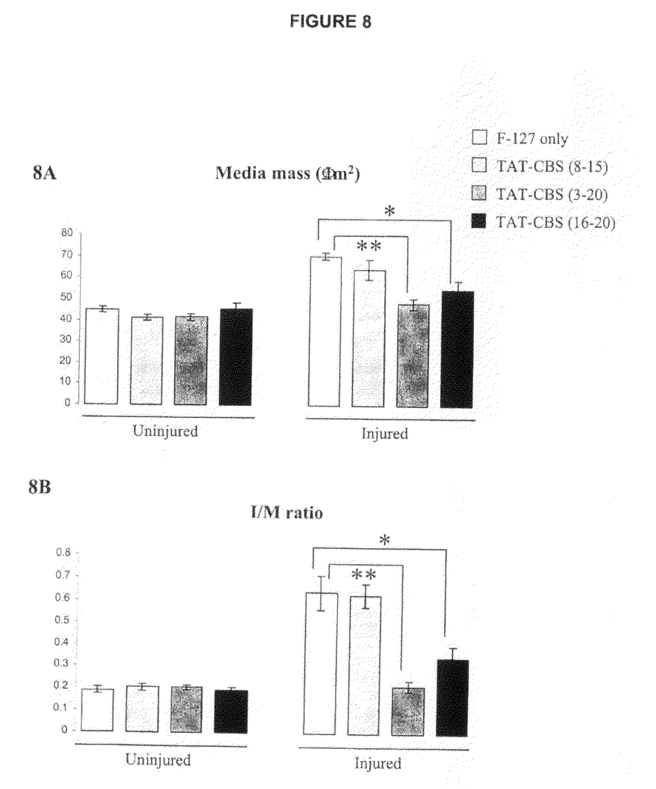

The application of TAT-SEQ ID NO:2(#3-20) and TAT-SEQ ID NO:2(#16-20) peptides had an anti-proliferative effect on the site of injury on mouse carotid arteries induced by wire denudation method (FIGS. 8A & B). These data showed in vivo anti-proliferative effects of specific fragments of CBS peptides (TAT-SEQ ID NO:2(#3-20) and TAT-SEQ ID NO:2(#16-20), in an animal model, providing for the location of the minimally active motif within SEQ ID NO:2

### Example 7

### CBS Peptides Reduce Proliferation of Tumour Spheres and Cancer Cells In Vivo

The effects of the CBS peptides derived from all or part of SEQ ID NO:2 on the growth of tumor-spheres, including mouse breast cancer models, are tested in vitro. Tumor-spheres are composed of a small collection of tumor-initiating cells in which the bioactivity and efficacy of anti-proliferative strategies are easily gauged. As described above, TAT-CBS peptides effectively reduce the level of proliferation of several mature cancer cells. Although malignantly transformed, the cell lines tested represented fully differentiated cell types. The TAT-CBS peptides also demonstrate a dose-dependent inhibition of the growth of tumor-initiating cells (i.e. in tumor-spheres) and inhibit the earliest phases of tumorigenesis, showing a high anti-cancer potential of CBS peptides.

In vivo studies employing standard intra-dermal and intra-peritoneal cancer models are conducted. In the latter, the effects of direct intra- vs. peri-tumor, and intra-peritoneal vs. intravenous routes of delivery of the TAT-CBS peptide at local- vs. total-body IC₅₀ concentrations determined to be effective in the tumor-sphere studies are shown. Cytotoxicity is followed by biochemical (serum and peritoneal LDH) and histological analyses. The growth of the in vivo tumor models is monitored by standard gravimetric and histopathological techniques.

### Example 8

### Investigations of the Anti-Proliferative Effect of TAT-CBS Using Tritiated-Thymidine Assays

To further establish that CBS works via an inhibitory effect on S-phase entry and to show that the effect was evident in human VSMC, DNA synthesis as measured by ³H-thymidine incorporation in human aortic VSMC treated with TAT-CBS (sequence RRRQRRKKRGGGAEFSARSRKRKANVTVFLQD) was examined compared to negative controls. Results showed that TAT-CBS, but not TAT-NC (sequence RRRQRRKKRGVDIDQARLKMLGQTRPHDDDDC) was able to produce a dose-dependent inhibitory effect on S-phase entry in human aortic VSMC (FIG. 7A, P<0.05 at 100 μM).

Additional experiments using the tritiated thymidine incorporation assay confirmed the results previously demonstrated in Example 2 using nucleofection experiments, that the proliferation of cyclin E1/E2-deficient mouse embryonic fibroblasts was not affected by treatment with CBS. The effects of TAT-CBS on S-phase entry were only observed in wild-type MEF (FIG. 7B, P<0.01 at 100 μM), but not cyclin E1^{-/-}E2^{-/-} MEF (FIG. 7C).

TABLE 1
Diagrammatic representation and sequence
of truncated peptides.

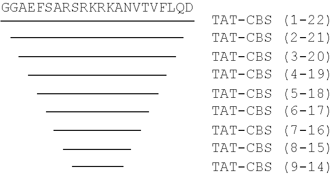

A

| Name | Sequence |
|---|---|
| TAT-CBS(1-22) | RRRQRRKKRGGGAEFSARSRKRKANVTVFLQD |
| TAT-CBS(2-21) | RRRQRRKKRGGAEFSARSRKRKANVTVFLQ |
| TAT-CBS(3-20) | RRRQRRKKRGAEFSARSRKRKANVTVFL |
| TAT-CBS(4-19) | RRRQRRKKRGEFSARSRKRKANVTVF |
| TAT-CBS(5-18) | RRRQRRKKRGFSARSRKRKANVTV |
| TAT-CBS(6-17) | RRRQRRKKRGSARSRKRKANVT |
| TAT-CBS(7-16) | RRRQRRKKRGARSRKRKANV |
| TAT-CBS(8-15) | RRRQRRKKRGRSRKRKAN |
| TAT-CBS(9-14) | RRRQRRKKRGSRKRKA |

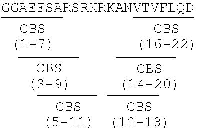

B

| Name | Sequence |
|---|---|
| CBS(1-7) | GGAEFSA |
| CBS(3-9) | AEFSARS |
| CBS(5-11) | FSARSRK |
| CBS(12-18) | RKANVTV |
| CBS(14-20) | ANVTVFL |
| CBS(16-22) | VTVFLQD |

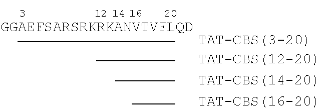

C

| Name | Sequence |
|---|---|
| TAT-CBS(3-20) | RRRQRRKKRGAEFSARSRKRKANVTVFL |
| TAT-CBS(12-20) | RRRQRRKKRGRKANVTVFL |
| TAT-CBS(14-20) | RRRQRRKKRGANVTVFL |
| TAT-CBS(16-20) | RRRQRRKKRGVTVFL |
| TAT-CBS(16-20, | RRRQRRKKRGLVFTV |
| scrambled) | |

D

| Name | Sequence |
|---|---|
| TAT only | RRRQRRKKRG |
| TAT-CBS(16-20) | RRRQRRKKRGVTVFL |
| TAT-CBS(16-20)K16 | RRRQRRKKRGKTVFL |
| TAT-CBS(16-20)K17 | RRRQRRKKRGVKVFL |
| TAT-CBS(16-20)K18 | RRRQRRKKRGVTKFL |
| TAT-CBS(16-20)K19 | RRRQRRKKRGVTVKL |
| TAT-CBS(16-20)K20 | RRRQRRKKRGVTVFK |

1. An isolated peptide comprising:
al all or part of the amino acid sequence: GGAEFSARSR KRKANVTVFL QD (SEQ ID NO:2), or
b) at least 80% sequence identity with the amino acid sequence of SEQ ID NO:2,
wherein the peptide reduces cell proliferation.

2. The peptide of claim 1, wherein the cell proliferation reduction activity comprises smooth muscle cell proliferation reduction activity or cancer cell proliferation reduction activity.

3. The peptide of claim 1, comprising three consecutive hydrophobic residues located within 5 amino acids of the C-terminus.

4. The peptide of claim 3, wherein at least one of the hydrophobic residues comprises a leucine residue.

5. The peptide of claim 1, comprising at least one leucine residue located within 5 amino acids of the C-terminus.

6. The peptide of claim 1, wherein the peptide comprises an amino acid sequence selected from the group consisting of SEQ ID NO:2, amino acids 2-21 of SEQ ID:2, amino acids 3-20 of SEQ ID NO:2, amino acids 4-19 of SEQ ID NO:2, amino acids 5-18 of SEQ ID NO:2, amino acids 6-17 of SEQ ID NO:2, amino acids 7-16 of SEQ ID NO:2, amino acids 8-15 of SEQ ID NO:2, amino acids 9-14 of SEQ ID NO:2, amino acids 12-20 of SEQ ID NO:2, amino acids 14-20 of SEQ ID NO:2, amino acids 16-20 of SEQ ID NO:2.

7. The peptide of claim 1, wherein the peptide comprises a fragment of 5-10, 10-15, 15-20 or 20-22 amino acids of the peptide of (SEQ ID NO:2), wherein the peptide reduces cell proliferation.

8. The peptide of claim 1, comprising at least: 5, 6, 7, 8, 9 or 10 amino acids of the peptide of SEQ ID NO:2.

9. (canceled)

10. The peptide of claim 1, wherein the peptide comprises all or part of the amino acid sequence of SEQ ID NO:3, SEQ ID NO:4 or SEQ ID NO:5.

11. The peptide of claim 1, wherein the peptide further comprises a TAT linker amino acid sequence comprising all or part of RRRQRRKKRG.

12. A pharmaceutical composition comprising the peptide of claim 1 and a carrier.

13. A method of reducing cell proliferation caused by cyclin E specific calcium/calmodulin dependent CDK2 activity in the cell, comprising administering to the cell the peptide of claim 1.

14. The method of claim 13, wherein the cell comprises a cancer cell or a smooth muscle cell.

15. The method of claim 14, wherein the cancer cell comprises a cervical cancer cell, an osteosarcoma cancer cell or a lung cancer cell.

16. (canceled)

17. A method of treatment of cancer in a mammal in need thereof, comprising administering to the mammal the peptide of claim 1.

18. The method of claim 17, wherein the cancer comprises cancer cells undergoing calcium sensitive cyclin E protein mediated cell proliferation.

19. The method of claim 17, wherein the cancer comprises cervical cancer, osteosarcoma or lung cancer.

20. (canceled)

21. A method of reducing proliferation of vascular smooth muscle cells in a mammal in need thereof, comprising administering to the mammal the peptide of claim 1.

22. The method of claim 21, wherein the peptide inhibits CDK2 activity by inhibiting the binding of calmodulin to cyclin E protein.

23. (canceled)

24. A method of treatment of a vaso-occlusive disorder in a mammal in need thereof comprising administering to the mammal the peptide of claim 1.

25. The method of claim 24 wherein the vaso-occlusive disorder comprises restenosis, Burger syndrome, atherosclerosis, scleroderma, Raynauds disease, hypertension, pulmonary hypertension or post-vascular surgery smooth muscle cell proliferation.

26. The method of claim 25, wherein the atherosclerosis comprises coronary artery disease, peripheral artery disease or cerebrovascular disease.

27. The method of claim 25, wherein the hypertension is caused by smooth muscle cell proliferation after vascular surgery.

28. The method of claim 25, wherein the vascular surgery is selected from the group consisting of coronary angioplasty, coronary stent placement, coronary by-pass surgery, peripheral stent placement, vascular grafting, thrombectomy, vascular angioplasty, and vascular stenting.

29. The method of claim 28, wherein the peptide or pharmaceutical composition is in a stent.

30. (canceled)

31. A method of treatment of a visceral smooth muscle cell disorder in a mammal in need thereof comprising administering to the mammal the peptide of claim 1.

32. The method of claim 31 wherein the visceral smooth muscle cell disorder comprises inflammatory bowel disease, bowel strictures, spastic bladder, urinary retention and uterine cramps.

33. (canceled)

34. (canceled)

35. An isolated nucleic acid (SEQ ID NO:1) encoding the peptide of claim 1.

36. An isolated antibody that selectively binds the peptide of claim 1.

37. A stent comprising the peptide of claim 1.
